(19) United States
(12) Patent Application Publication
(10) Pub. No.: US 2006/0110395 A1
(43) Pub. Date: May 25, 2006

(54) Use of CRKD as a breast cancer marker and cancer therapy target

(76) Inventors: Gerald R. Crabtree, Woodside, CA (US); Kevin Corbit, San Francisco, CA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/191,457

(22) Filed: Jul. 28, 2005

Related U.S. Application Data
(60) Provisional application No. 60/591,653, filed on Jul. 28, 2004.

(51) Int. Cl. G01N 33/574; A61K 39/395; C07K 16/30

(52) U.S. Cl. 424/155.1; 435/7.23; 530/388.8

(57) ABSTRACT

In certain aspects, the present invention discloses the use of CRKD as a marker for a cell-proliferative disorder, preferably as a marker for breast cancer. The invention discloses antibodies and fragments thereof which specifically bind CRKD, methods of diagnosing, methods for assessing CRKD status, methods of monitoring the efficacy of a treatment, and kits for the detection of a CRKD marker. The invention also discloses the use of CRKD and CRKR as targets for treating cell-proliferative disorders, preferably as targets breast cancer treatment. The invention further discloses methods for identifying and isolating mammary stem cells.

### CROSS REFERENCE TO RELATED APPLICATIONS

This application claims the benefit of priority of U.S. Provisional Application No. 60/591,653 filed Jul. 28, 2004. The teachings of the referenced Provisional Application are incorporated herein by reference in their entirety.

### FEDERALLY SPONSORED RESEARCH OR DEVELOPMENT

Certain work described herein was funded by the National Institute of Health. The United States government may have certain rights in this invention.

### BACKGROUND OF THE INVENTION

Breast cancer is a leading cause of death in women. While the pathogenesis of breast cancer is unclear, transformation of normal breast epithelium to a malignant phenotype may be the result of genetic factors.

Regardless of its origin, breast cancer morbidity increases significantly if a lesion is not detected early in its progression. Thus, considerable effort has focused on the elucidation of early cellular events surrounding transformation in breast tissue. Such effort has led to the identification of several potential breast cancer markers. For example, alleles of the BRCA1 and BRCA2 genes have been linked to hereditary and early-onset breast cancer. Wooster et al., Science, 265: 2088-2090 (1994). The wild-type BRCA1 allele encodes a tumor suppressor protein. Deletions and/or other alterations in that allele have been linked to transformation of breast epithelium. Accordingly, detection of mutated BRCA1 alleles or their gene products has been proposed as a means for detecting breast, as well as ovarian, cancers. However, BRCA1 is limited as a cancer marker because BRCA1 mutations fail to account for the majority of breast cancers. Ford et al., British J. Cancer, 72: 805-812 (1995). Similarly, the BRCA2 gene, which has been linked to forms of hereditary breast cancer, accounts for only a small portion of total breast cancer cases. Ford et al., supra.

Several other genes have been linked to breast cancer and may serve as markers for the disease, either directly or via their gene products. Such potential markers include the TP53 gene and its gene product, the p53 tumor suppressor protein. Malkin et al., Science, 250: 1233-1238 (1990). The loss of heterozygosity in genes such as the ataxia telangiectasia gene has also been linked to a high risk of developing breast cancer. Swift et al., N. Engl. J. Med., 325: 1831-1836 (1991). A problem associated with many of the markers proposed to date is that the oncogenic phenotype is often the result of a gene deletion, thus requiring detection of the absence of the wild-type form as a predictor of transformation.

There is, therefore, a need in the art for specific, reliable markers that are differentially expressed in normal and transformed breast tissue and that may be useful in the diagnosis of breast cancer or in the prediction of its onset. Such markers and methods for their use are provided herein.

The use of genetic screens in model organisms has been a remarkably powerful and productive approach to the understanding of fundamental aspects of development. However, this approach has been difficult to apply to certain processes in vertebrates. For example the development of the breast is specific to mammals and hence the evolutionary origin of this organ might have required the creation of new signaling mechanisms not present in lower vertebrates. One such pathway is NFAT signaling which was initially discovered in T lymphocytes (1-4) and conveys signals to the nucleus after triggering the T cell receptor, a vertebrate-specific receptor. The four genes that encode the cytoplasmic subunits of NFAT transcription complexes (NFATc genes) are found only in vertebrates and indeed are not present even in the genomes of primitive invertebrates such as Ciona Intestinalis (5). Analysis of mice with mutation of the different subunits of NFAT transcription complexes have indicated that this pathway is used widely in mammalian development. NFAT signaling is critical not only for development of a recombinational immune system, but also a vascular system, the myocardium, heart valves, and cartilage and bone [see (6) for review]. A particularly interesting example occurs in the vertebrate nervous system where NFAT signaling specifically conveys signals from receptors for axonal guidance molecules such as neurotrophins, netrins and others where it regulates the rate of axonal extension needed for the longer axonal trajectories of larger organisms (7). In these systems NFAT signaling appears to serve the needs of receptors and ligands, such as neurotrophins and the T cell receptor that are also specific for vertebrates. These observations indicate that NFAT signaling might play essential roles in the development of other vertebrate-specific organs such as the breast.

NFAT signaling is initiated by several classes of receptors, including both tyrosine kinase and non-tyrosine kinase receptors as well as the Wnt and Fas receptors. In addition, several Ca²⁺ channels, such as the NMDA receptor, L type channels, and CRAC channels can initiate NFAT signaling. These receptors lead to an influx of Ca²⁺ and activation of calcineurin phosphatase activity. Calcineurin then dephosphorylates and leads to the nuclear entry of the cytoplasmic subunits of NFAT transcription complexes (NFATc proteins). Once in the nucleus NFAT complexes are formed on DNA by combination of the different cytoplasmic subunits and nuclear subunits. Since NFAT proteins have a weak DNA binding domain they need a nuclear partner (NFATn) for binding to DNA. This requirement is the basis for the role of this pathway in signal integration and coincidence detection between signals coming from distinct pathways (8).

Remarkably, null mutations for the different NFATc genes have given essentially identical phenotypes as the null mutations for calcineurin b (Cnb) that disrupt its activity, indicating that at least in the developing mouse this pathway is relatively unbranched and that calcineurin is dedicated to the dephosphorylation of the NFATc proteins (7, 12, 39). The biochemical basis of this specificity is probably the unconventionally tight binding of calcineurin to the NFATc proteins via two different interaction domains in the N-termini of the NFATc family members (9-11). Thus, unlike most kinases and phosphatases, calcineurin is sequestered to its specific substrate.

### SUMMARY OF THE INVENTION

One aspect of the invention relates to a method for augmenting diagnosis of a cell-proliferative disorder comprising detecting the presence of a CRKD marker in a biological sample obtained from a patient, wherein the presence of said marker is indicative of cancer. For example, the cell-proliferative disorder is breast cancer. In certain cases, the sample is a breast tissue sample or a body fluid sample (e.g., blood or serum).

In certain embodiments of the method, the CRKD marker is a CRKD polypeptide. To illustrate, a CRKD polypeptide is encoded by a nucleic acid comprising SEQ ID NO:1 or SEQ ID NO: 3 or a fragment thereof, or is encoded by a nucleic acid that hybridizes to SEQ ID NO:1 or SEQ ID NO: 3 under stringent conditions, or comprises the amino acid sequence of SEQ ID NO: 2 or SEQ ID NO: 4 or a fragment thereof. Optionally, the fragment comprises the extracellular domain of a CRKD polypeptide.

In certain embodiments of the method, the presence of said CRKD polypeptide or fragment thereof is determined by: (a) contacting said sample with a binding moiety which binds specifically to said CRKD polypeptide or fragment thereof to produce a binding moiety-CRKD polypeptide complex, and (b) detecting the binding moiety-CRKD polypeptide complex, wherein the presence of said complex is indicative of breast cancer. For example, the binding moiety is an antibody or a fragment thereof. The antibody includes, but is not limited to, a monoclonal antibody and a polyclonal antibody. Optionally, the antibody further comprises a label, such as a label selected from the group consisting of a radioactive label, a hapten label, a fluorescent label, a chemiluminescent label, a spin label, a colored label, and an enzymatic label. In certain embodiments, the method further comprises the step of measuring the concentration of the polypeptide in the sample.

In certain embodiments of the method, the CRKD marker is a nucleic acid. For example, the nucleic acid encodes a CRKD polypeptide. Optionally, the nucleic acid is detected by a nucleic acid probe, such as a probe in a microarray. In certain case, a microarray further comprises a nucleic acid probe which specifically binds to a CRKR marker.

Another aspect of the invention relates to a method for assessing CRKD status in a patient comprising detecting the presence of a CRKD marker in a biological sample obtained from the patient. Optionally, the method further comprises quantifying the amount of the CRKD marker in the biological sample, wherein the quantity of CRKD marker in the sample is indicative of CRKD status. For example, the sample is a breast tissue sample or a body fluid sample (e.g., blood or serum).

In certain embodiments of the method, the CRKD marker is a CRKD polypeptide. Optionally, the fragment comprises the extracellular domain of a CRKD polypeptide.

In other embodiments of the method, the CRKD marker is a nucleic acid. For example, the nucleic acid encodes a CRKD polypeptide. Optionally, the nucleic acid is detected by a nucleic acid probe, such as a probe in a microarray.

Another aspect of the invention relates to an isolated antibody or fragment thereof which binds specifically to a CRKD polypeptide. To illustrate, a CRKD polypeptide is encoded by a nucleic acid comprising SEQ ID NO:1 or SEQ ID NO: 3 or a fragment thereof, or comprises the amino acid sequence of SEQ ID NO: 2 or SEQ ID NO: 4 or a fragment thereof. Optionally, the antibody or fragment thereof binds specifically to the extracellular domain of said CRKD polypeptide. In certain cases, the antibody or fragment thereof further comprises a label, selected from the group consisting of a fluorescent label, a radiolabel, a toxin, a metal compound and biotin. Examples of the fluorescent label include Texas Red, phycoerythrin (PE), cytochrome c, and fluorescent isothiocyante (FITC). Examples of the radiolabel include 32P, 33P, 43K, 47Sc, 52Fe, 57Co, 64Cu, 67Ga, 67Cu, 68Ga, 71Ge, 75Br, 76Br, 77Br, 77As, 77Br, 81Rb/81MKr, 87MSr, 90Y, 97Ru, 99Tc, 100Pd, 101Rh, 103Pb, 105Rh, 109Pd, 11Ag, 111In, 113In, 119Sb, 121Sn, 123I, 125I, 127Cs, 128Ba, 129Cs, 131I, 131Cs, 143Pr, 153Sm, 161Tb, 166Ho, 169Eu, 177Lu, 186Re, 188Re, 189Re, 191Os, 193Pt, 194Ir, 197Hg, 199Au, 203Pb, 211At, 212Pb, 212Bi and 213Bi. Examples of the toxin include ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin. Optionally, the antibody or fragment thereof is a CRKD antagonist.

Another aspect of the invention relates to an isolated antibody or fragment thereof which binds specifically to a CRKR polypeptide. To illustrate, the CRKR polypeptide is encoded by the nucleic acid sequence of SEQ ID NO:5 or a fragment thereof, or comprises the amino acid sequence of SEQ ID NO:6 or a fragment thereof. Optionally, the antibody or fragment thereof is a CRKD antagonist. In certain cases, the antibody of fragment thereof further comprises a label, selected from the group consisting of a fluorescent label, a radiolabel, a toxin, a metal compound and biotin.

Another aspect of the invention relates to a kit for detecting a cell-proliferative disorder comprising: (a) a receptacle for receiving a biological sample; (b) a first binding moiety which binds specifically to a CRKD marker; and (c) a reference sample. In certain cases, the first binding moiety comprises a label. Optionally, the kit further comprises a second binding moiety which selectively binds to the first binding moiety. Similarly, the second binding moiety may optionally comprise a label.

Another aspect of the invention relates to a CRKD antagonist. For example, the antagonist binds specifically to a CRKD polypeptide (e.g., the extracellular domain of a CRKD polypeptide). Optionally, the antagonist inhibits the binding of CRKD to CRKR.

Another aspect of the invention relates to a method of monitoring the effectiveness of a treatment against a cell-proliferative disorder in which CRKD is upregulated, comprising quantifying the amount of a CRKD marker in a biological sample, wherein a decrease in the CRKD marker is indicative of the effectiveness of the treatment. For example, the cell-proliferative disorder is breast cancer.

Another aspect of the invention relates to a method of treating a cell-proliferative disorder in which CRKD is upregulated comprising administering to a mammal an effective amount of the antibody or fragment thereof which binds specifically to a CRKD polypeptide. For example, the cell-proliferative disorder is breast cancer. Optionally, such method further comprises administering a chemotherapeutic agent.

Another aspect of the invention relates to a method of treating a cell-proliferative disorder in which CRKD is upregulated comprising administering to a mammal an effective amount of pharmaceutical composition comprising a CRKD antagonist. For example, the cell-proliferative disorder is breast cancer. Optionally, such method further comprises administering a chemotherapeutic agent.

Another aspect of the invention relates to a method of treating a cell-proliferative disorder in which CRKD is upregulated comprising administering to a mammal an effective amount of a pharmaceutical composition comprising a calcium channel agonist. For example, the cell-proliferative disorder is breast cancer. Optionally, such method further comprises administering a chemotherapeutic agent.

Another aspect of the invention relates to a method of treating a cell-proliferative disorder comprising modulating the expression of a CRKD polypeptide or a CRKR polypeptide in a mammal. For example, the cell-proliferative disorder is breast cancer. Optionally, modulating the expression of a CRKD or a CRKR polypeptide comprises contacting a cell with a nucleic acid selected from the group consisting of a siRNA probe, an antisense nucleic acid or a ribozyme.

Another aspect of the invention relates to a method of conducting a business comprising: a) obtaining a sample; b) detecting the presence of a CRKD marker in the sample; and c) reporting the results of such detection. Optionally, the method further comprises quantifying the amount of CRKD marker in the sample.

Another aspect of the invention relates to a method to identify the presence of mammary stem cells in a mixed cell population, comprising detecting the presence of a CRKD marker, wherein the presence of CRKD polypeptide is indicative of the presence of mammary stem cells in a mixed cell population.

Another aspect of the invention relates to a method for isolating mammary stem cells comprising: a) obtained a mixed cell population; b) exposing said mixed cell population to a binding moiety specific for a CRKD marker; and c) separating the cells bound to the binding moiety, thereby isolating mammary stem cells.

Another aspect of the invention relates to a micrroarray comprising one or more probes for detecting a CRKD marker. Optionally, the microarray further comprises one or more probes for detecting a CRKR marker.

Another aspect of the invention relates to use of a composition comprising a CRKD antagonist in the manufacture of a medicament for treating a cell-proliferative disorder.

Another aspect of the invention relates to use of a composition comprising an agent that modulates the expression of CRKD or CRKR in the manufacture of a medicament for treating a cell-proliferative disorder.

Another aspect of the invention relates to use of a composition comprising a calcium channel agonist in the manufacture of a medicament for treating a cell-proliferative disorder.

Another aspect of the invention relates to a method of screening for CRKD antagonists, comprising: a) contacting a CRKD polypeptide with a test compound; b) determining whether the test compound binds the CRKD polypeptide; and c) further determining whether the test compound inhibits the binding of CRKD to CRKR, wherein a test compound that binds the CRKD polypeptide and inhibits the binding of CRKD to CRKR is a CRKD antagonist. Optionally, the method further comprises determining whether the test compound binds the extracellular domain of said CRKD polypeptide.

Another aspect of the invention relates to a method to screen for a compound used to treat a cell-proliferate disorder comprising: a) identifying a CRKD antagonist: and b) determining whether said CRKD antagonist is effective against a cell-proliferative disorder.

### BRIEF DESCRIPTION OF THE DRAWINGS

FIG. 1 sets out the nucleotide and amino acid sequence of CRKD, a calcineurin-regulated gene. 2539-bp transcript and putative open reading frame (single letter amino acid code). On the right, the protein is represented graphically, with colored segments corresponding to the underline colors of the amino acid sequence. Red, hydrophobic signal sequence; yellow, kringle domain; green, transmembrane region.

FIG. 2A illustrates that CRKD is transmembrane protein. (Left panel) 5×10⁵ 293T cells were transfected with buffer (mock), 1 μg pCRKD/HA, or 1 μg of pcDNA-LacZ-V5/His; 48 hours later cell lysates were collected, separated by 12.5% SDS-PAGE, and immunoblotted for HA-containing proteins as described below. The membrane was stripped and re-probed with action to demonstrate equal loading. (Right panel) 5×10⁵ 293T cells were transfected with buffer (mock) or 1 μg pCRKD(EC)/His. 48 hours later cell lysates (L) and conditioned medium (CM) were collected, separated by 12.5% SDS-PAGE, and immunoblotted for 6× His-containing proteins as described in below. The position of molecular weight markers (BioRad) is shown on the right of each panel.

FIG. 2B illustrates that CRKD is upregulated in Cnb-null embryos. Whole E9.5 embryo were collected from a (Cnb^{+/Δ}×Cnb^{+/Δ}) cross and homogenized in RIPA buffer as described in methods. The corresponding yolk sacs were used for genotyping as described in Methods. Proteins were separated by 12.5% SDS-PAGE, and immunoblotted for CRKD as described in Methods. The membrane was then stripped and re-probed with anti-Cnb and anti-P actin antibodies to demonstrate genotypes and equal loading, respectively.

FIG. 3A illustrates that CRKD is expressed in the developing mammary buds. Whole-mount in situ analysis was performed on E12.5 CD 1 embryos as described in Methods. The left panel is a control showing the results using the sense (CRKD-S) riboprobe, while the middle panel was hybridized with the antisense (CRKD-AS) riboprobe. The right panel is a high magnification picture showing the #2 and #3 mammary bud epithelial staining in detail.

FIG. 3B illustrates that CRKD is repressed during mammary differentiation. Northern blot analysis to detect CRKD using 10 μg of total RNA per lane from mammary glands at various stages (the numbers indicate the days of that stage) as described in below. The lower figure is a picture of the ethidium bromide-stained gel to demonstrate equal loading (28S and 18S RNA shown). To confirm this result at the protein level, a virgin, day eight of lactation (L8), and day four of involution (I4) #4 mammary gland were homogenized separately in RIPA. 20 μg of protein lysates were separated by 12.5% SDS-PAGE, and immunoblotted for CRKD as described in Methods. The membrane was then stripped, and reprobed with anti-β actin antibodies to demonstrate equal loading. Finally, to ensure the Northern blot analysis was not a result of dilution the CRKD RNA, in situ hybridization was performed on paraformaldehyde-fixed, formalin-embedded sections as described in Methods, Both a sense (CRKD-S) and antisense (CRKD-AS) ribroprobe was used. The sections of the virgin gland are shown at 400× magnification to show detail (CRKD message is detected as brown against a blue hematoxylin counterstain), while the lactating (L8) and involuting (I4) sections are shown at 200× to demonstrate expression from a larger portion of the section.

FIG. 4A illustrates that CRKD is specifically secreted from human breast cancer lines. Cell lysates and conditioned medium were prepared from primary human mammary epithelial cells (HMEC) or one of three breast cancer lines (MCF7, MDA-MB-231, MCF10A) as described in Methods. 20 μg of protein lysates were separated by 12.5% SDS-PAGE and immunoblotted for CRKD as described below. The membrane was then stripped and re-probed with anti-β actin antibodies to demonstrate equal loading. Alternatively, the conditioned medium (˜20× concentrated) from a single well of a 6-well plate was used and immunoblotted for CRKD.

FIG. 4B illustrates that CRKD is found in the serum of breast cancer patients. One milliliter of freshly-obtained sera from ten women with metastatic breast cancer (Breast cancer, 1-10, upper panel) and ten women with no history of disease (Normal, 11-20, bottom panel) were immunoprecipitated with anti-CRKD as described in Methods. Bound proteins were separated by 12.5% SDS-PAGE and immunoblotted for CRKD as described below.

FIG. 5A illustrates the expression cloning of CRKR, a putative CRKD binding partner. FIG. 5A illustrate a T7 phage screen for CRKD binding partners. A T7 phage breast cancer cDNA library was screened with purified CRKD(EC)His on ELISA plates. Subsequently, bound phage were plated, transferred to nitrocellulose, and screened for CRKD(EC)His binding by ‘Far Western’ with anti-His antibodies as described below. The arrow in the far left panel shows a positive-binding phage following one round of screening, which was picked, amplified, and subjected to further screening. The middle panel shows the majority of phage binding CRKD(EC)His after the fourth round of screening. Sixteen positive-binding phage were picked and subjected to PCR amplification. The results reveal a ˜180-bp insert.

FIG. 5B shows the nucleotide and amino acid sequence of CRKR, a CRKD binding protein. Part of the 3465-bp transcript and putative open reading frame (single letter amino acid code). On the right, the protein is represented graphically, with colored segments corresponding to the underline colors of the amino acid sequence. Red, hydrophobic signal sequence; yellow, Ig-like domain; green, transmembrane region.

### DETAILED DESCRIPTION OF THE INVENTION

I. Overview

A variety of membrane receptors and Ca²⁺ channels transduce signals to the nucleus via the calcineurin-induced dephosphorylation of the cytoplasmic subunits of NFAT transcription complexes. Studies of mice lacking components of this signaling pathway indicate that it plays critical roles in mammalian development. Based on the ending that genes encoding the cytoplasmic subunits of NFAT complexes are only found in the genomes of higher vertebrates, we have screened for target genes in vertebrate-specific mammary gland formation. This approach lead us to identify a previously unrecognized kringle domain-containing protein (CRKD) that is actively repressed by calcineurin-NFATc signaling in the embryo. CRKD is a single transmembrane protein expressed in the developing mammary buds of E12.5 mice. In the adult animal, CRKD is specifically expressed in the immature mammary gland and is repressed during functional differentiation. CRKD is over-expressed in primary breast cancer and breast cancer cell lines, and is shed from breast cancer cells but not primary human mammary epithelial cells. Soluble CRKD is found in the serum of some breast cancer patients suggesting that CRKD might be a marker for, or involved in the pathogenesis of this disease. Finally, we report the identification and cloning of a novel transmembrane binding partner for CRKD, CRKR, which could have a role in hetero- and/or homotypic cell-cell signaling in the developing mammary gland. The disclosed and claimed methods are the direct result of these findings.

II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

As used herein, hybridization under “stringent conditions” include conditions equivalent to about 20-27° C. below the melting temperature (Tm) of the DNA duplex formed in about 1 M salt. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

“Specifically binds” or “binds specifically to” means that the binding agent binds to the antigen on the target cell with greater affinity than it binds unrelated antigens. Preferably such affinity is at least 10-fold greater, more preferably at least 100-fold greater, and most preferably at least 1000-fold greater than the affinity of the binding agent for unrelated antigens.

As used herein a “binding moiety” refers to any molecule that specifically binds to a target molecule. A binding moiety may comprise a ligand, an antibody, a nucleic acid, a protein, a peptide, a peptidomimetic, or other molecule.

The term “antibody” as used herein is intended to include whole antibodies, e.g., of any isotype (IgG, IgA, IgM, IgE, etc), and includes fragments thereof which also specifically bind to a protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies. The term also includes humanized antibodies and chimeric antibodies.

The antibodies and peptides of the present invention may be labeled. As used herein, “label” is used to mean a detectable label which is used to visualize the binding of an antibody to its target protein or receptor. Alternatively, antibodies and peptides of the present invention may be labeled with a radiolabel, an iron-related compound, or a toxin which would kill the cell to which it binds. Radiolabels and toxins are well known in the art and include, for example, ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶ Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi, ricin, ricin A chain (ricin toxin), Pseudomonas exotoxin (PE), diphtheria toxin (DT), Clostridium perfringens phospholipase C (PLC), bovine pancreatic ribonuclease (B PR), pokeweed antiviral protein (PAP), abrin, abrin A chain (abrin toxin), cobra venom factor (CVF), gelonin (GEL), saporin (SAP), modeccin, viscumin and volkensin. Iron-related compounds include, for example Fe₂O₃ and Fe₃O₄.

The term “recombinant” as used in reference to a nucleic acid indicates any nucleic acid that is positioned adjacent to one or more nucleic acid sequences that it is not found adjacent to in nature. A recombinant nucleic acid may be generated in vitro, for example by using the methods of molecular biology, or in vivo, for example by insertion of a nucleic acid at a novel chromosomal location by homologous or non-homologous recombination. The term “recombinant” as used in reference to a polypeptide indicates any polypeptide that is produced by expression and translation of a recombinant nucleic acid.

As used herein a “portion” or “fragment” of a protein or of an amino acid sequence denotes a contiguous peptide comprising, in sequence, at least ten amino acids from the protein or amino acid sequence (e.g. amino acids 1-10, 34-43, or 127-136 of the protein or sequence). Preferably, the peptide comprises, in sequence, at least twenty amino acids from the protein or amino acid sequence. More preferably, the peptide comprises, in sequence, at least forty amino acids from the protein or amino acid sequence.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides.

“Operably linked” is intended to mean that the nucleotide sequence is linked to a regulatory sequence in a manner which allows expression of the nucleotide sequence. Regulatory sequences are art-recognized and are selected to direct expression of the subject peptide. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990).

The term “gene construct” refers to a vector, plasmid, viral genome or the like which includes a coding sequence, can transfect cells, preferably mammalian cells, and can cause expression of the antibody, antigen binding fragment, peptide or peptidomimetic of the cells transfected with the construct.

The term “amino acid residue” is known in the art. In general the abbreviations used herein for designating the amino acids and the protective groups are based on recommendations of the IUPAC-IUB Commission on Biochemical Nomenclature (see Biochemistry (1972) 11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagines, lysine, arginine, praline, histidine, phenylalanine, tyrosine, and tryptophan.

The term “amino acid residue” further includes analogs, derivatives and congeners of any specific amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino aid analog such as, for example, cyanoalanine, canavanine, djenkolic acid, norleucine, 3-phosphoserine, homoserine, dihydroxy-phenylalanine, 5-dydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included as the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) and (L) sterioisomers. D- and L-α-Amino acids are represented by the following Fischer projections and wedge-and-dash drawings. In the majority of cases, D- and L-amino acids have R- and S-absolute configurations, respectively.

Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into non-peptide compounds with the activity of the parent peptides.

Moreover, as is apparent from the present disclosure, mimetopes of the subject antibodies, antigen binding fragments, peptides, and peptidomimetics can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988, p 123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9^{th} American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modified (Roark et al. in Peptides: Chemistry and Biology, G. R. Marshall ed., ESCOM Publisher: Leiden, Netherlands, 1988, p 134). Also, see generally, Session III: Analytic and synthetic methods, in Peptides: Chemistry and Biology, G. R. Marshall ed., ESCOM Publisher: Leiden, Netherlands, 1988).

Each of the embodiments of the present invention can be used as a composition when combined with a pharmaceutically acceptable carrier or excipient. “Carrier” and “excipient” are used interchangeably herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” is defined herein as a carrier that is physiologically acceptable to the administered patient and that retains the therapeutic properties of the antibodies. Pharmaceutically-acceptable carriers and their formulations are well-known and generally described in, for example, Remington's Pharmaceutical Sciences (18^{th} Edition, ed. A. Gennaro, Mack Publishing Co., Easton, Pa., 1990). On exemplary pharmaceutically acceptable carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antibodies from the administration site of one organ, or portion of the body, to another organ, or portion of the body. Each carrier must be “acceptable” in the sense of being compatible with the other ingredients of the formulation and not injurious to the patient. Nor should a pharmaceutically acceptable carrier alter the specific activity of the antibodies. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “cell-proliferative disorder” denotes malignant as well as nonmalignant populations of transformed cells which morphologically often appear to differ from the surrounding tissue.

As used herein, “transformed cells” refers to cell which have spontaneously converted to a state of unrestrained growth, i.e., they have acquired the ability to grow through an indefinite number of divisions in culture. Transformed cells may be characterized by such terms as neoplastic, anaplastic and/or hyperplastic with respect to their loss of growth control.

As used herein, the term “cancer” is used to mean a condition in which a cell in a patient's body undergoes abnormal, uncontrolled proliferation. Thus, “cancer” is a cell-proliferative disorder. Non-limiting examples of cancers include breast cancer, cervical cancer, prostate cancer, colon cancer, lung cancer, skin cancer, leukemia, lymphoma, lupus, melanoma or any other type of cancer.

“Administering” is defined herein as a means providing the composition to the patient in a manner that results in the composition being inside the patient's body. Such an administration can be by any route including, without limitation, subcutaneous, intradermal, intravenous, intra-arterial, intraperitoneal, and intramuscular.

By “treating” a patient or subjecting a patient to “treatment”, it is meant that the patient's symptoms are partially or totally alleviated, or remain static following treatment according to the invention. A patient that has been treated can exhibit a partial or total alleviation of symptoms (for example, tumor load). The term “treatment” is intended to encompass prophylaxis, therapy and cure.

A “therapeutically effective amount” is defined herein an effective amount of composition for producing some desire therapeutic effect.

A physician or veterinarian having ordinary skill in the art can readily determine and prescribe the “effective amount” (ED₅₀) of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The term “sample” is defined herein as blood, blood product, biopsy tissue, serum, and any other type of fluid or tissue that can be extracted from a patient or a mammal. The terms “sample” and “biological sample” are used interchangeably in this application.

As used herein, a “patient” can be any mammal.

By “assessing CRKD status” it is meant detecting of the CRKD marker in a sample. The detection of a CRKD marker in a sample may be useful in the diagnosis or prognosis of a disease or condition.

By “augmenting diagnosis” it is meant diagnosing, aiding in the diagnosis of, or contributing to the diagnosis of, a particular disease or condition.

III. Compositions

A. CRKD Polypeptides and Nucleic Acids

CRKD polypeptides, or nucleic acids encoding CRKD polypeptides, or portions thereof may act as markers useful in the detection of a cell-proliferative disorder, the monitoring of a cell-proliferative disorder or as targets for treating a cell-proliferative disorder. In a preferred embodiment, CRKD is used as a breast cancer marker.

As used herein a “CRKD marker” refers to a CRKD polypeptide or a nucleic acid (such as an mRNA) encoding a CRKD polypeptide.

As used herein the term “CRKD polypeptide” refers to the full-length CRKD polypeptide, or fragment thereof. Thus, the term “CRKD polypeptide” includes fragments of CRKD such as the extracellular domain of CRKD or soluble CRKD.

In one embodiment, the CRKD polypeptide of the invention is encoded by SEQ ID NO:1 (GenBank Accession No. AY522649), or a fragment thereof.

In another embodiment, the CRKD polypeptide of the invention is encoded by a nucleic acid that hybridizes to SEQ ID NO:1 under stringent conditions.

In another embodiment, the CRKD polypeptide comprises the amino acid sequence of SEQ ID NO:2 (GenBank Accession No. AAS13454), or a fragment thereof. Soluble CRKD consists of amino acids 1-166 of SEQ ID NO:2.

In another embodiment, the CRKD polypeptide comprises an amino acid sequence having conservative amino acid substitutions as compared to SEQ ID NO:2, or a fragment of said amino acid sequence.

In a preferred embodiment, the CRKD polypeptide is a human polypeptide and is encoded by SEQ ID NO:3 (GenBank Accession No. NM_{—}052880), or a fragment thereof.

In another preferred embodiment, the CRKD polypeptide is encoded by a nucleic acid that hybridizes to SEQ ID NO:3 under stringent conditions.

In another preferred embodiment, the CRKD polypeptide comprises the amino acid sequence of SEQ ID NO:4 (GenBank Accession No. NP_{—}443112), or a fragment thereof.

In another embodiment, the CRKD polypeptide comprises an amino acid sequence having conservative amino acid substitutions as compared to SEQ ID NO:4, or a fragment of said amino acid sequence.

B. CRKR Polypeptides and Nucleic Acids

As used herein the term “CRKR polypeptide” includes fragments of a CRKR polypeptide.

In one embodiment, the CRKR polypeptide of the invention is encoded by SEQ ID NO:5 (GenBank Accession No. AY522648) or a fragment thereof.

In another embodiment, the CRKR polypeptide is encoded by a nucleic acid that hybridizes to SEQ ID NO:5 under stringent conditions.

In another embodiment, the CRKR polypeptide comprises the amino acid sequence of SEQ ID NO:6 (GenBank Accession No. AAS 13453), or a fragment thereof.

In another embodiment, the CRKR polypeptide comprises an amino acid sequence having conservative amino acid substitutions as compared to SEQ ID NO:6, or a fragment of said amino acid sequence.

C. Variants of CRKD and/or CRKR

The claimed invention includes the use of variants of the CRKD and CRKR polypeptides. Variants of the present invention may have an amino acid sequence that is different by one or more amino acid substitutions to the amino acid sequence disclosed in SEQ ID NOS: 2, 4, or 6. Embodiments which comprise amino acid deletions and/or additions are also contemplated. The variant may have conservative changes (amino acid similarity), wherein a substituted amino acid has similar structural or chemical properties, for example, the replacement of leucine with isoleucine. Guidance in determining which and how many amino acid residues may be substituted, inserted, or deleted without abolishing biological or proposed pharmacological activity may be reasonably inferred in view of this disclosure and may further be found using computer programs well known in the art, for example, DNAStar® software.

Amino acid substitutions may be made, for instance, on the basis of similarity in polarity, charge, solubility, hydrophobicity, hydrophilicity, and/or the amphipathic nature of the residues as long as a biological and/or pharmacological activity of the native molecule is retained.

Negatively charged amino acids include aspartic acid and glutamic acid; positively charged amino acids include lysine and arginine; amino acids with uncharged polar head groups having similar hydrophilicity values include leucine, isoleucine, and valine; amino acids with aliphatic head groups include glycine, alanine; asparagine, glutamine, serine; and amino acids with aromatic side chains include tryptophan, phenylalanine, and tyrosine.

Example substitutions are set forth in Table 1 as follows:

TABLE 1

| Original Residue | Example conservative substitutions |
|---|---|
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; Ile; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

“Homology” is a measure of the identity of nucleotide sequences or amino acid sequences. In order to characterize the homology, subject sequences are aligned so that the highest percentage homology (match) is obtained, after introducing gaps, if necessary, to achieve maximum percent homology. N- or C-terminal extensions shall not be construed as affecting homology. “Identity” per se has an art-recognized meaning and can be calculated using published techniques. Computer program methods to determine identity between two sequences, for example, include DNAStar® software (DNAStar Inc. Madison, Wis.); the GCG® program package (Devereux, J., et al. Nucleic Acids Research (1984) 12(1): 387); BLASTP, BLASTN, FASTA (Atschul, S. F. et al., J. Molec Biol (1990) 215: 403). Homology (identity) as defined herein is determined conventionally using the well-known computer program, BESTFIT® (Wisconsin Sequence Analysis Package, Version 8 for Unix, Genetics Computer Group, University Research Park, 575 Science Drive, Madison, Wis., 53711). When using BESTFIT® or any other sequence alignment program (such as the Clustal algorithm from MegAlign software (DNAStar®) to determine whether a particular sequence is, for example, about 90% homologous to a reference sequence, according to the present invention, the parameters are set such that the percentage of identity is calculated over the full length of the reference nucleotide sequence or amino acid sequence and that gaps in homology of up to about 90% of the total number of nucleotides in the reference sequence are allowed.

Ninety percent of homology is therefore determined, for example, using the BESTFIT® program with parameters set such that the percentage of identity is calculated over the full length of the reference sequence, and wherein up to 10% of the amino acids in the reference sequence may be substituted with another amino acid. Percent homologies are likewise determined, for example, to identify preferred species, within the scope of the claims appended hereto. As noted above, N- or C-terminal extensions shall not be construed as affecting homology. Thus, when comparing two sequences, the reference sequence is generally the shorter of the two sequences. This means that, for example, if a sequence of 50 nucleotides in length with precise identity to a 50 nucleotide region within a 100 nucleotide polynucleotide is compared, there is 100% homology as opposed to only 50% homology.

Although a naturally occurring CRKD or CKKR polypeptide and a variant polypeptide may only possess 90% identity, they are actually likely to possess a higher degree of similarity, depending on the number of dissimilar codons that are conservative changes. Conservative amino acid substitutions can frequently be made in a protein without altering either the conformation or function of the protein. Similarity between two sequences includes direct matches as well a conserved amino acid substitutes which possess similar structural or chemical properties, e.g., similar charge as described in Table 1.

Percentage similarity (conservative substitutions) between two polypeptides may also be scored by comparing the amino acid sequences of the two polypeptides by using programs well known in the art, including the BESTFIT program, by employing default settings for determining similarity.

In one embodiment, the CRKD polypeptide is a variant of SEQ ID NO:2 or SEQ ID NO:4. In one embodiment, the CRKD polypeptide is at least 95%, 90%, 85% or 80% homologous to SEQ ID NO:2 or SEQ ID NO:4. In another embodiment, the CRKD polypeptide is encoded by a nucleic acid that is at least 95%, 90%, 85% or 80% homologous to SEQ ID NO:1 or SEQ ID NO:3

In one embodiment the CRKR polypeptide is a variant of SEQ ID NO:6. In another embodiment, the CRKR polypeptide is at least 95%, 90%, 85% or 80% homologous to SEQ ID NO:6. In another embodiment the CRKR polypeptide is encoded by a nucleic acid that is at least 95%, 90%, 85% or 80% homologous to SEQ ID NO:5.

D. CRKD Antagonists

The present invention also encompasses CRKD antagonists. As used herein, a “CRKD antagonist” is any molecule which inhibits the biological or functional effect of naturally occurring CRKD. A CRKD antagonist may inhibit the biological or functional effect of naturally occurring CRKD by any means.

In one embodiment, a CRKD antagonist inhibits the biological or functional effect of naturally occurring CRKD by decreasing the expression of CRKD.

In another embodiment, a CRKD antagonist inhibits the biological or functional effect of naturally occurring CRKD by specifically binding to CRKD.

A CRKD antagonist may be a peptide or a peptidomimetic of CRKD.

A CRKD antagonist may also be an antibody or fragment thereof that binds CRKD or CRKR.

In one embodiment, the invention comprises a CRKD antagonist which binds specifically to CRKD. In another embodiment, the invention comprises a CRKD antagonist which binds specifically to the extracellular domain of a CRKD polypeptide. In another embodiment, the invention comprises a CRKD antagonist which binds specifically to soluble CRKD. In one embodiment, the CRKD antagonist inhibits the binding of CRKD to CRKR.

In another embodiment, the invention comprises a CRKD antagonist which binds specifically to CRKR.

E. Antibodies to CRKD and CRKR Polypeptides

Another aspect of the invention pertains to an antibody which specifically binds to a CRKD or a CRKR polypeptide.

In one embodiment, the invention comprises an isolated antibody or fragment thereof which binds specifically to a CRKD polypeptide. In one embodiment, the antibody or fragment thereof binds specifically to a CRKD polypeptide encoded by a nucleic acid comprising SEQ ID NO:1 or SEQ ID NO:3, or comprising the amino acid sequence of SEQ ID NO:2 or SEQ ID NO:4. In another embodiment, the antibody or fragment thereof binds specifically to the extracellular domain of a CRKD polypeptide. In another soluble CRKD.

In one embodiment, the invention comprises an isolated antibody or fragment thereof which binds specifically to a CRKR polypeptide. In one embodiment, the CRKR polypeptide is encoded by the nucleic acid sequence of SEQ ID NO:5 or comprises the amino acid sequence of SEQ ID NO:6.

In one embodiment, the invention comprises an isolated antibody or fragment thereof which is a CRKD antagonist.

In one embodiment, the antibody or fragment thereof further comprises a label, wherein the label is selected from the group consisting of a fluorescent label, a radiolabel, a toxin, a metal compound and biotin. In one embodiment the fluorescent label is selected from the group consisting of Texas Red, phycoerythrin (PE), cytochrome c, and fluorescent isothiocyante (FITC). In another embodiment, the radiolabel is selected from the group consisting of ³²P, ^{33P,43}K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹ Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. In another embodiment, the toxin is selected from the group consisting of ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin.

A person of skilled in the art would know how to make antibodies or fragments thereof which specifically bind to a CRKD or CRKR polypeptide. For example, by using peptides based on the sequence of the subject proteins, specific antisera or monoclonal antibodies can be made using standard methods. Chickens, or a mammal such as a mouse, a hamster or rabbit can be immunized with an immunogenic form of the peptide (e.g., an antigenic fragment which is capable of eliciting an antibody response). Techniques for conferring immunogenicity on a protein or peptide include conjugation to carriers or other techniques well known in the art. For instance, a peptidyl portion of one of the subject proteins can be administered in the presence of adjuvant. The progress of immunization can be monitored by detection of antibody titers in plasma or serum. Standard ELISA or other immunoassays can be used with the immunogen as antigen to assess the levels of antibodies.

Following immunization, antisera can be obtained and, if desired, polyclonal antibodies against the target protein can be further isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, Nature, 256: 495-497, 1975), as well as the human B cell hybridoma technique (Kozbar et al., Immunology Today, 4: 72, 1983), and the EBV-hybridoma technique to produce human monoclonal antibodies (Cole et al., Monoclonal Antibodies and Cancer Therapy, Alan R. Liss, Inc. pp. 77-96, 1985). Hybridoma cells can be screened immunochemically for production of antibodies specifically reactive with the CRKD or CRKR polypeptides and the monoclonal antibodies isolated.

The term antibody as used herein is intended to include fragments thereof which are also specifically reactive with one of the subject proteins or complexes including the subject proteins. Antibodies can be fragmented using conventional techniques and the fragments screened for utility in the same manner as described above for whole antibodies. For example, F(ab′)2 fragments can be generated by treating antibody with pepsin. The resulting F(ab′)2 fragment can be treated to reduce disulfide bridges to produce Fab′ fragments. The antibody of the present invention is further intended to include bispecific and chimeric molecules, as well as single chain (scFv) antibodies.

The subject antibodies include trimeric antibodies and humanized antibodies, which can be prepared as described, e.g., in U.S. Pat. No. 5,585,089. Also within the scope of the invention are single chain antibodies. All of these modified forms of antibodies as well as fragments of antibodies are intended to be included in the term “antibody” and are included in the broader term “binding moiety”.

Antibodies of the present invention can be made recombinantly. Linkers may be added to the nucleic acid sequences of the heavy and light chains to increase flexibility of the antibody. In the case of a scFv, the linkers are added to connect the VH and VL chains and the varying composition can effect solubility, proteolytic stability, flexibility, and folding. In a preferred embodiment, a linker of the present invention has the amino sequence GSTSG. In a preferred embodiment, a linker of the present invention has the amino sequence GGSSRSS. Linkers are well-known in the art and can comprise varied amino acid residues depending on the flexibility needed in the resulting recombinant protein to allow for biological activity.

F. Peptides and Peptidomimetics

One embodiment of the present inventions are peptides, and compositions thereof, which may be used to detect a CRKD polypeptide. Peptides of the present invention can comprise 5-50 amino acid residues. More preferably, peptides of the present invention comprise 5-30 amino acid residues. More preferably, peptides of the present invention comprise 5-20 amino acid residues. More preferably, peptides of the present invention comprise 10-15 amino acid residues.

Another aspect of the invention provides a peptide or peptidomimetic, e.g., wherein one or more backbone bonds are replaced or one or more side chains of a naturally occurring amino acid are replaced with sterically and/or electronically similar functional groups.

In certain embodiments, the peptide or peptidomimetic is formulated in a pharmaceutically acceptable excipient.

G. Compositions

Each of the embodiments of the present invention can be used as a composition when combined with a pharmaceutically acceptable carrier or excipient. Pharmaceutically acceptable carriers are physiologically acceptable and retain the therapeutic properties of the antibodies or peptides present in the composition. Pharmaceutically-acceptable carriers are well-known and generally described in, for example, Remington's Pharmaceutical Sciences (18^{th} Edition, ed. A. Gennaro, Mack Publishing Co., Easton, Pa., 1990). On exemplary pharmaceutically acceptable carrier is physiological saline.

H. Labels

The antibodies, antigen binding fragments, and peptides of the present invention may be associated with a toxin, a radionuclide, an iron-related compound, or a chemotherapeutic agent which would be toxic when delivered to a cancer cell.

The antibodies, antigen binding fragments, and peptides of the present invention may be associated with detectable label, such as a radionuclide, iron-related compound, or a fluorescent agent for immunodetection of target antigens.

The antibodies and peptides of the present invention which are immunoreactive with the VAG domain of provasopressin can be labeled with a detectable label, such as a radiolabel, a toxin, or fluorescent label

Non-limiting examples of radiolabels include, for example, ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi.

Non-limiting examples of toxins include, for example, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin.

Non-limiting examples of fluorescent labels include, for example, FITC, Texas Red, phycoerythrin (PE), and cytochrome c.

Non-limiting examples of iron-related compounds include, for example, magnetic iron-oxide particles, ferric or ferrous particles, Fe₂O₃, and Fe₃O₄. Iron-related compounds and methods of labeling antibodies and polypeptides can be found, for example, in U.S. Pat. Nos. 4,101,435 and 4,452,773, and U.S. published applications 20020064502 and 20020136693, all of which are hereby incorporated by reference in their entirety.

Additionally, other labels, such as biotin followed by streptavidin-alkaline phosphatase (AP), horseradish peroxidase (HRP) are contemplated by the present invention.

Methodology for labeling proteins, such as antibodies, antigen binding fragments, and peptides are well known in the art. When the antibodies, antigen binding fragments, and peptides of the present invention are labeled with a radiolabel or toxin, the antibodies, antigen binding fragments, and peptides can be prepared as pharmaceutical compositions which are useful for therapeutic treatment of patients exhibiting increased levels of provasopressin wherein the pharmaceutical compositions are administered to the patient in an effective amount.

I. Chemotherapeutic Agents

Chemotherapeutic agents contemplated by the present invention include chemotherapeutic drugs that are commercially available.

Merely to illustrate, the chemotherapeutic can be an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and/or a DNA repair inhibitor.

Chemotherapeutic agents may be categorized by their mechanism of action into, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors. Preferred dosages of the chemotherapeutic agents are consistent with currently prescribed dosages.

J. Linkers

It may be necessary in some instances to introduce an unstructured polypeptide linker region between a label of the present invention and portions of the antibodies, antigen binding fragments, peptides, or peptidomimetics. The linker can facilitate enhanced flexibility, and/or reduce steric hindrance between any two fragments. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the 1cI and LexA proteins. Alternatively, the linker can be of synthetic origin. For instance, the sequence (Gly₄Ser)₃ can be used as a synthetic unstructured linker. Linkers of this type are described in Huston et al. (1988) PNAS 85:4879; and U.S. Pat. No. 5,091,513, both incorporated by reference herein.

Within the linker, the amino acid sequence may be varied based on the preferred characteristics of the linker as determined empirically or as revealed by modeling. For instance, in addition to a desired length, modeling studies may show that side groups of certain amino acids may interfere with the biological activity, e.g. DNA binding or transcriptional activation, of the protein. Considerations in choosing a linker include flexibility of the linker, charge of the linker, and presence of some amino acids of the linker in the naturally-occurring subunits. The linker can also be designed such that residues in the linker contact DNA, thereby influencing binding affinity or specificity, or to interact with other proteins. For example, a linker may contain an amino acid sequence which can be recognized by a protease so that the activity of the chimeric protein could be regulated by cleavage. In some cases, particularly when it is necessary to span a longer distance between subunits or when the domains must be held in a particular configuration, the linker may optionally contain an additional folded domain.

In some embodiments it is preferable that the design of a linker involve an arrangement of domains which requires the linker to span a relatively short distance, preferably less than about 10 Angstroms (Å). However, in certain embodiments, depending, e.g., upon the selected domains and the configuration, the linker may span a distance of up to about 50 Angstroms.

K. Toxins

In certain embodiments, the subject antibodies, antigen binding fragments, peptides and peptidomimetics can be covalently or non-covalently coupled to a cytotoxin or other cell proliferation inhibiting compound, in order to localize delivery of that agent to a tumor cell. For instance, the agent can be selected from the group consisting of alkylating agents, enzyme inhibitors, proliferation inhibitors, lytic agents, DNA or RNA synthesis inhibitors, membrane permeability modifiers, DNA intercalators, metabolites, dichlorethylsulfide derivatives, protein production inhibitors, ribosome inhibitors, inducers of apoptosis, and neurotoxins.

Chemotherapeutics useful as active moieties which when conjugated to antibodies, antigen binding fragments, peptides and peptidomimetics of the present invention are specifically delivered to tumorigenic cells are typically, small chemical entities produced by chemical synthesis. Chemotherapeutics include cytotoxic and cytostatic drugs. Chemotherapeutics may include those which have other effects on cells such as reversal of the transformed state to a differentiated state or those which inhibit cell replication. Examples of known cytotoxic agents useful in the present invention are listed, for example, in Goodman et al., The Pharmacological Basis of Therapeutics, Sixth Edition, A. G. Gilman et al, eds./Macmillan Publishing Co. New York, 1980. These include taxanes, such as paclitaxel (Taxol®) and docetaxel (Taxotere®); nitrogen mustards, such as mechlorethamine, cyclophosphamide, melphalan, uracil mustard and chlorambucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkoloids, such as vinblastine and vincristine; antibiotics, such as dactinomycin, daunorubicin, doxorubicin, bleomycin, mithramycin and mitomycin; enzymes, such as L-asparaginase; Platinum coordination complexes, such as cisplatin; substituted urea, such as hydroxyurea; methyl hydrazine derivatives, such as procarbazine; adrenocortical suppressants, such as mitotane; hormones and antagonists, such as adrencortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

Drugs that interfere with intracellular protein synthesis can also be used; such drugs are known to those skilled in the art and include puromycin, cycloheximide, and ribonuclease.

Most of the chemotherapeutic agents currently in use in treating cancer possess functional groups that are amenable to chemical cross-linking directly with an amine or carboxyl group of an agent of the present invention. For example, free amino groups are available on methotrexate, doxorubicin, daunorubicin, cytosinarabinoside, bleomycin, gemcitabine, fludarabine, and cladribine while free carboxylic acid groups are available on methotrexate, melphalan, and chlorambucil. These functional groups, that is free amino and carboxylic acids, are targets for a variety of homobifunctional and heterobifunctional chemical cross-linking agents which can crosslink these drugs directly to a free amino group of an antibody, antigen binding fragment, peptide or peptidomimetics.

Peptide and polypeptide toxins are also useful as active moieties, and the present invention specifically contemplates embodiments wherein the antibodies, antigen biding fragments, peptides and peptidomimetics of the present invention are coupled to a toxin. In certain preferred embodiments, the antibodies, antigen binding fragments, peptides and peptidomimetics and toxin are both polypeptides and are provided in the form of a fusion protein. Toxins are generally complex toxic products of various organisms including bacteria, plants, etc. Examples of toxins include but are not limited to: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVR), gelonin (GEL), saporin (SAP), modeccin, viscumin and volkensin.

The invention further contemplates embodiments in which the antibodies, antigen binding fragments, peptides and peptidomimetics are coupled to a polymer or a functionalized polymer (e.g., a polymer conjugated to another molecule). Preferred examples include water soluble polymers, such as, polyglutamic acide or polyaspartic acide, conjugated to a drug such as a chemotherapeutic or antiangiogenic agent, including, for example, paclitaxel or docetaxel.

In certain preferred embodiments, particularly where the cytotoxic moiety is chemically cross-linked to the antibody, antigen biding fragment, peptide and peptidomimetic moieties, the linkage is hydrolysable, e.g., such as may be provided by use of an amide or ester group in the linking moiety.

In certain embodiments, the subject antibodies, antigen binding fragments, peptides and peptidomimetics can be coupled with an agent useful in imaging tumors. Such agents include: metals, metal chelators; lanthanides; lanthanide chelators; radiometals; radiometal chelators; positron-emitting nuclei; microbubbles (for ultrasound); liposomes; molecules microencapsulated in liposomes or nanosphere; monogrystalline iron oxide ananocompounds; magnetic resonance imaging contrast agents; light absorbing, reflecting and/or scattering agents; colloidal particules; fluorophores, such as near-infrared fluorophores. In many embodiments, such secondary functionality will be relatively large, e.g., at least 25 amu in size, and in many instances can be at least 50, 100 or 250 amu in size.

In certain preferred embodiments, the secondary functionality is a chelate moiety for chelating a metal, e.g., a chelator for a radionuclide useful for radiotherapy or imaging procedures.

Radionuclides useful within the present invention include gamma-emitters, positron-emitters, Auger electron-emitters, X-ray emitters and fluorescence-emitters, with beta- or alpha-emitters preferred for therapeutic use. Examples of radionuclides useful as toxins in radiation therapy include: ³²P, ³³P, ⁴³K, ⁴⁷Sc, ⁴²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho or ¹⁷⁷Lu. Conditions under which a chealator will coordinate a metal are described, for example, by Gansow et al., U.S. Pat. Nos. 4,831,175, 4,454,106 and 4,472,509. Within the present invention, “radionuclide” and “radiolabel” are used interchangeably.

^{99m}Tc is a particularly attractive radioisotope for diagnostic applications, as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain preferred embodiments, the modified antibodies, antigen binding fragments, peptides and peptidomimetics include a chelating agent for technium.

In still other embodiments, the secondary functionality can be a radiosensitizing agent, e.g., a moiety that increases the sensitivity of cells to radiation. Examples of radiosensitizing agents include netroimidazoles, metronidazole and misonidazole (see: DeVita, V. T. Jr. in Harrison's Principles of Internal Medicine, p. 68, McGraw-Hill Book Co., N.Y. 1983, which is incorporated herein by reference). The modified antibodies, antigen biding fragments, peptides and peptidomimetics that comprise a radiosensitizing agent as the active moiety are administered and localize at the target cell. Upon exposure of the individual to radiation, the radiosensitizing agent is “excited” and causes the death of the cell.

There are a wide range of moieties which can serve as chelators and which can be derivatized to the antibodies, antigen biding fragements, peptides and peptidomimetics of the present invention. For instance, the chelator can be a derivative of 1,4,7,10-tetraazacyclododecanetetraacetic acide (DOTA), ethylenediaminetetraacetic acid (EDTA), diethylenetriaminepentaacetic acide (DTPA) and 1-p-Isothiocyanato-benzyl-methyl-diethylenetriaminepentaacetic acid (ITC-MX). These chelators typically have groups on the side chain by which the chelator can be used for attachment to subject antibodies, antigen binding fragments, peptides and peptidomimetics. Such groups include, e.g., benzylisothiocyanate, by which the DOTA, DTPA or EDTA can be coupled to, e.g., an amine group.

In one embodiment, the chelate moiety is an “NₓS_{y}” chelate moiety. As defined herein, the term “NₓS_{y} chelates” includes bifunctional chelators that are capable of coordinately binding a metal or radiometal and, preferably, have N₂S₂ or N₃S cores. Exemplary NₓS_{y} chelates are described, e.g., in Fritzberg et al. (1988) PNAS 85:4024-29; and Weber et al. (1990) Bioconjugate Chem. 1:431-37; and in the references cited therein.

The Jacobsen et al. PCT application WO 98/12156 provides methods and compositions, i.e. synthetic libraries of binding moieties, for identifying compounds which bind to a metal atom. The approach described in that publication can be used to identify binding moieties which can subsequently be added to antibodies, antigen binding fragments, peptides and peptidomimetics to derive the modified antibodies, antigen binding fragments, peptides and peptideomimetics of the present invention.

A problem frequently encountered with the use of conjugate proteins in radiotherapeutic and radio diagnostic applications is a potentially dangerous accumulation of the radiolabeled moiety fragments in the kidney. When the conjugate is formed using a acid- or base-labile linker, cleavage of the radioactive chelate from the protein can advantageously occur. If the chelate is of relatively low molecular weight, as most of the subject modified antibodies, antigen binding fragments, peptides and peptidomimetics are expected to be, it is not retained in the kidney and is excreted in the urine, thereby reducing the exposure of the kidney to radioactivity. However, in certain instances, it may be advantageous to utilize acid- or base-labile linkers in the subject ligands for the same reasons they have been used in labeled proteins.

Accordingly, certain of the subject labeled/modified antibodies, antigen binding fragments, peptides and peptidomimetics can be synthesized, by standard methods known in the art, to provide reactive functional groups which can form acid-labile linkages with, e.g., a carbonyl group of the ligand. Examples of suitable acid-labile linkages include hydrazone and thiosemicarbazone functions. These are formed by reacting the oxidized carbohydrate with chelates bearing hydrazide, thiosemicarbazide, and thiocarbazide functions, respectively.

Alternatively, base-cleavable linkers, which have been used for the enhanced clearance of the radiolabel from the kidneys, can be used. See, for example, Weber et al. 1990 Bioconjug. Chem. 1:431. The coupling of a bifunctional chelate to antibodies, antigen binding fragments, peptides and peptidomimetics via a hydrazide linkage can incorporate base-sensitive ester moieties in a linker spacer arm. Such an ester-containing linker unit is exemplified by ethylene glycolbis (succinimidyl succinate), (EGS, available from Pierce Chemical Co., Rockford, Ill.), which has two terminal N-hydroxysuccinimide (NHS) ester derivatives of two 1,4-dibutyric acid units, each of which are linked to a single ethylene glycol moity by two alkyl esters. One NHS ester may be replaced with a suitable amine-containing BFC (for example 2-aminobenzyl DTPA), while the other NHS ester is reacted with a limiting amount of hydrazine. The resulting hydrazide is used for coupling to the antibodies, antigen binding fragments, peptides and peptidomimetcs, forming an ligand-BFC linkage containing two alkyl ester functions. Such a conjugate is stable at physiological pH, but readily cleaved at basic pH.

Antibodies, antigen binding fragments, peptides and peptidomimetics labeled by chelation are subject to radiation-induced scission of the chelator and to loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotope in the urine.

In still other embodiments, the antibodies, antigen binding fragments, peptides and peptidomimetics are coupled to a Boron addend, such as a carborane. For example, carboranes can be prepared with carboxyl functions on pendant side chains, as is well known in the art. Attachment of such carboranes to an amine functionality, e.g., as may be provided on the antibodies, antigen binding fragments, peptides and peptidomimetics, can be achieved by activation of the carboxyl groups of the carboranes and condensation with the amine group to produce the conjugate. Such modified antibodies, antigen binding fragments, peptides and peptidomimetics can be used for neutron captive therapy.

The present invention also contemplates the modification of the subject peptides with dyes, for example, useful in photodynamic therapy, and used in conjunction with appropriate non-ionizing radiation. The use of light and porphyrins in methods of the present invention is also contemplated and their use in cancer therapy has been reviewed by van den Bergh, Chemistry in Britain, 22: 430-437 (1986), which is incorporated by reference herein in its entirety.

One embodiment of the present invention includes antibodies, antigen binding fragments thereof, peptides, and peptidomimetics labeled with a fluorescent label. Common fluorescent labels include, for example, FITC, PE, Texas Red, cytochrome c, etc. Techniques for labeling polypeptides and proteins are well-known in the art.

One embodiment of the present invention includes antibodies, antigen binding fragments thereof, peptides, and peptidomimetics labeled with a metal compound, such as iron which can be used in MRI imaging and/or for treatment. Iron-containing compounds include both ferrous and ferric-containing compounds, such as ferric-oxides. Specific examples include Fe₂O₃ and Fe₃O₄. Iron-containing compounds and methods of making iron-coupled antibodies and fragments thereof are described in U.S. Pat. Nos. 4,101,435 and 4,452,773 and published U.S. patent applications 20020064502 and 20020136693, all of which are hereby incorporated by reference in their entireties.

IV. Methods of Augmenting Diagnosis

The invention provides a method for augmenting diagnosis of a cell-proliferative disorder in a patient comprising detecting the presence of a CRKD marker in a sample, wherein the presence of said marker is indicative of the cell-proliferative disorder. In one embodiment, the cell-proliferative disorder is cancer. In another embodiment, the cancer is breast cancer, cervical cancer, prostate cancer, colon cancer, lung cancer, skin cancer, leukemia, lymphoma, lupus, melanoma or any other type of cancer. In one embodiment the cancer is breast cancer.

The invention also provides a method for assessing CRKD status in a patient comprising detecting the presence of a CRKD marker in a biological sample obtained from a patient. In one embodiment, the method for assessing CRKD status further comprising quantifying the amount of CRKD marker in the biological sample, wherein the amount of CRKD marker in the biological sample is indicative of CRKD status.

The CRKD marker can be any of the markers described above. In one embodiment, the CRKD marker is a CRKD polypeptide or a fragment thereof. In a preferred embodiment, the marker is the extracellular domain of CRKD or soluble CRKD.

In one embodiment the CRKD marker is a CRKD polypeptide encoded by a nucleic acid comprising SEQ ID NO:1 or SEQ ID NO:3 or a fragment thereof. In another one embodiment the CRKD marker is a polypeptide encoded by a nucleic acid that hybridizes to SEQ ID NO:1 or SEQ ID NO:3 under stringent conditions.

In another embodiment, the CRKD marker is a CRKD polypeptide which comprises the amino acid sequence of SEQ ID NO:2 or SEQ ID NO:4 or a fragment thereof. In one embodiment, the CRKD marker is the extracellular domain of a CRKD polypeptide or soluble CRKD.

The methods of the present invention may be performed in any relevant sample. A sample can be a tissue, a cell or a body fluid. In one embodiment, the tissue is breast tissue, preferably breast biopsy tissue. The body fluid can be any body fluid, including but not limited to blood, serum, plasma, urine, saliva, sputum and breast ductal secretions. In one embodiment, the body fluid is blood or serum.

A. Protein Based Assays

In one embodiment, the CRKD marker is a CRKD polypeptide or a fragment thereof. In one embodiment, the detected fragment is the extracellular fragment of a CRKD polypeptide or soluble CRKD.

A CRKD polypeptide may be detected using any assay method available in the art, a subset of which is discussed below. Non-limiting examples of such methods include immunohistochemistry, ELISAs, MRI and Western blots.

In one embodiment the presence of CRKD polypeptide marker is determined by: (a) contacting said sample with a binding moiety which binds specifically to said CRKD polypeptide or fragment thereof to produce a binding moiety-CRKD polypeptide complex, and (b) detecting the binding moiety-CRKD polypeptide complex, wherein the presence of said complex is indicative of breast cancer.

In one embodiment, the binding moiety is an antibody or a fragment thereof. In one embodiment, the antibody is a monoclonal antibody. In another embodiment, the antibody is a polyclonal antibody. In another embodiment the antibody further comprises a label. In one embodiment, the label is selected from the group consisting of a radioactive label, a hapten label, a fluorescent label, a chemiluminescent label, a spin label, a colored label, and an enzymatic label. In one embodiment, the method for detecting the presence of a CRKD polypeptide further comprises the step of measuring the concentration of the polypeptide in the sample.

In one embodiment, the protein may be reacted with a binding moiety, such as an antibody, capable of specifically binding the protein being detected. Binding moieties, such as antibodies, may be designed using methods available in the art so that they interact specifically with the protein being detected. Optionally, a labeled binding moiety may be utilized. In such an embodiment, the sample is reacted with a labeled binding moiety capable of specifically binding the protein, such as a labeled antibody, to form a labeled complex of the binding moiety and the target protein being detected. Detection of the presence of the labeled complex then may provide an indication of the presence of a breast cancer in the individual being tested.

In one approach, for example, the marker protein may be detected using a binding moiety capable of specifically binding the marker protein. The binding moiety may comprise, for example, a member of a ligand-receptor pair, i.e., a pair of molecules capable of having a specific binding interaction. The binding moiety may comprise, for example, a member of a specific binding pair, such as antibody-antigen, enzyme-substrate, nucleic acid-nucleic acid, protein-nucleic acid, protein-protein, or other specific binding pair known in the art. Binding proteins may be designed which have enhanced affinity for a target protein. Optionally, the binding moiety may be linked with a detectable label, such as an enzymatic, fluorescent, radioactive, phosphorescent or colored particle label. The labeled complex may be detected, e.g., visually or with the aid of a spectrophotometer or other detector.

A CRKD may be detected using any of a wide range of immunoassay techniques available in the art. For example, the skilled artisan may employ the sandwich immunoassay format to detect breast cancer in a body fluid sample. Alternatively, the skilled artisan may use conventional immuno-histochemical procedures for detecting the presence of CRKD polypeptide a tissue sample using one or more labeled binding proteins.

In a sandwich immunoassay, two antibodies capable of binding the marker protein generally are used, e.g., one immobilized onto a solid support, and one free in solution and labeled with a detectable chemical compound. Examples of chemical labels that may be used for the second antibody include radioisotopes, fluorescent compounds, spin labels, colored particles such as colloidal gold and colored latex, and enzymes or other molecules that generate colored or electrochemically active products when exposed to a reactant or enzyme substrate. When a sample containing the marker protein is placed in this system, the marker protein binds to both the immobilized antibody and the labeled antibody, to form a “sandwich” immune complex on the support's surface. The complexed protein is detected by washing away non-bound sample components and excess labeled antibody, and measuring the amount of labeled antibody complexed to protein on the support's surface. Alternatively, the antibody free in solution, which can be labeled with a chemical moiety, for example, a hapten, may be detected by a third antibody labeled with a detectable moiety which binds the free antibody or, for example, the hapten coupled thereto.

Both the sandwich immunoassay and tissue immunohistochemical procedures are highly specific and very sensitive, provided that labels with good limits of detection are used. A detailed review of immunological assay design, theory and protocols can be found in numerous texts in the art, including Butt, W. R., ed. (1984) Practical Immunology, Marcel Dekker, N.Y. and Harlow et al. eds. (1988) Antibodies, A Laboratory Approach, Cold Spring Harbor Laboratory.

In general, immunoassay design considerations include preparation of antibodies (e.g., monoclonal or polyclonal antibodies) having sufficiently high binding specificity for the target protein to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the target protein. The higher the antibody binding specificity, the lower the target protein concentration that can be detected.

Antibodies to an isolated CRKD polypeptide which are useful in assays for detecting a cancer in an individual may be generated using standard immunological procedures well known and described in the art. See, for example, Practical Immunology, Butt, N. R., ed., Marcel Dekker, NY, 1984. Briefly, an isolated target protein is used to raise antibodies in a xenogeneic host, such as a mouse, goat or other suitable mammal. The marker protein is combined with a suitable adjuvant capable of enhancing antibody production in the host, and is injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (e.g., cell-free emulsion). Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Butt (1984) (supra).

In addition, genetically engineered biosynthetic antibody binding sites, also known in the art as BABS or sFv's, may be used in the practice of the instant invention. Methods for making and using BABS comprising (i) non-covalently associated or disulfide bonded synthetic VH and VL dimers, (ii) covalently linked VH-VL single chain binding sites, (iii) individual VH or VL domains, or (iv) single chain antibody binding sites are disclosed, for example, in U.S. Pat. Nos. 5,091,513; 5,132,405; 4,704,692; and 4,946,778. Furthermore, BABS having requisite specificity for the CRKD polypeptide can be derived by phage antibody cloning from combinatorial gene libraries (see, for example, Clackson et al. (1991) Nature 352: 624-628; or U.S. Pat. No. 5,837,500). Briefly, phage each expressing on their coat surfaces BABS having immunoglobulin variable regions encoded by variable region gene sequences derived from mice pre-immunized with CRKD polypeptide, or fragments thereof, are screened for binding activity against immobilized CRKD polypeptide. Phage which bind to the immobilized CRKD polypeptide are harvested and the gene encoding the BABS is sequenced. The resulting nucleic acid sequences encoding the BABS of interest then may be expressed in conventional expression systems to produce the BABS protein.

Marker proteins may also be detected using gel electrophoresis techniques available in the art. In two-dimensional gel electrophoresis, the proteins are separated first in a pH gradient gel according to their isoelectric point. The resulting gel then is placed on a second polyacrylamide gel, and the proteins separated according to molecular weight (see, for example, O'Farrell (1975) J. Biol. Chem. 250: 4007-4021; or Berkelman et al. (October 1998) 2-D Electrophoresis Using Immobilized pH Gradients: Principles and Methods, Amersham Pharmacia Biotech Pub. 80-6429-60, Rev. A).

One or more marker proteins may be detected by first isolating proteins from a sample obtained from an individual suspected of having breast cancer, and then separating the proteins by two-dimensional gel electrophoresis to produce a characteristic two-dimensional gel electrophoresis pattern. The pattern may then be compared with a standard gel pattern produced by separating, under the same or similar conditions, proteins isolated from normal or cancer cells. The standard gel pattern may be stored in, and retrieved from an electronic database of electrophoresis patterns. The presence of a CRKD polypeptide in the two-dimensional gel provides an indication that the sample being tested was taken from a person with cancer, particularly breast cancer. As with the other detection assays described herein, the detection of two or more proteins, for example, in the two-dimensional gel electrophoresis pattern further enhances the accuracy of the assay. The assay thus permits the early detection and treatment of cancer.

Mass spectrometry may also be used to detect a marker protein. Preferred mass spectrometry methods include MALDI-TOF mass spectrometry and MALDI-TOF using derivatized chip surfaces (SELDI). Useful mass spectrometry methods for detecting a marker protein are described, for example, in the Examples and in U.S. Pat. Nos. 5,719,060; 6,124,137; 6,207,370; 6,225,047; 6,281,493; and 6,322,970.

These detection methods may be used in combination with each other, with other detection methods, and/or with one or more purification methods to reduce the complexity of a biological sample. Thus, for example, proteins isolated by two-dimensional gel electrophoresis could be probed with an antibody that specifically binds the marker protein, or could be assayed by mass spectrometry. Similarly, as described in the Examples, a biological sample may be subjected to biochemical fractionation prior to analysis by mass spectrometry or by other techniques such as gel electrophoresis and/or immunoassays. A marker protein may also be detected indirectly, for example, by subjecting it to enzymatic treatment, and subsequently detecting the products of that treatment.

B. Neucleic Acid Based Assays

In another embodiment, the CRKD marker is a nucleic acid encoding CRKD or a fragment thereof. A nucleic acid encoding CRKD can be detected using any method available in the art of subset of which is discussed below.

In one embodiment, the presence of a CRKD nucleic acid marker is detected by a nucleic acid probe which may be designed using standard methods and are used to identify DNA or mRNA encoding CRKD. See, e.g., Maniatis et al., Molecular Cloning: A Laboratory Manual, Cold Spring Harbor Press (1989). In one embodiment, the nucleic acid probe is complementary to at least a portion of a DNA or RNA encoding a CRKD polypeptide.

In one embodiment, the nucleic acid probe capable of detecting CRKD is in a microarray containing a plurality of probes. In one embodiment, the nucleic acid probe capable of detecting CRKD is in a microarray that further comprises a nucleic acid probe specific to CRKR.

A detecting step according to the invention may comprise amplifying nucleic acid encoding a CRKD polypeptide using a polymerase chain reaction (“PCR”) or a reverse-transcriptase polymerase chain reaction. Detection of products of the PCR may be accomplished using known techniques, including hybridization with nucleic acid probes complementary to the amplified sequence.

Gene probes comprising complementary RNA or, preferably, DNA to CRKD nucleotide sequences or mRNA sequences encoding CRKD polypeptides may be produced using established recombinant techniques or oligonucleotide synthesis. The probes hybridize with complementary nucleic acid sequences presented in the test specimen, and can provide exquisite specificity. A short, well-defined probe, coding for a single unique sequence is most precise and preferred. Larger probes are generally less specific. While an oligonucleotide of any length may hybridize to an mRNA transcript, oligonucleotides typically within the range of 8-100 nucleotides, preferably within the range of 15-50 nucleotides, are envisioned to be most useful in standard hybridization assays. Choices of probe length and sequence allow one to choose the degree of specificity desired. Hybridization is carried out at from 50° to 65° C. in a high salt buffer solution, formamide or other agents to set the degree of complementarity required. Furthermore, the state of the art is such that probes can be manufactured to recognize essentially any DNA or RNA sequence. For additional particulars, see, for example, Berger et al. (1987) Guide to Molecular Techniques (Methods of Enzymology, vol. 152).

A wide variety of different labels coupled to the probes or antibodies may be employed in the assays. The labeled reagents may be provided in solution or coupled to an insoluble support, depending on the design of the assay. The various conjugates may be joined covalently or noncovalently, directly or indirectly. When bonded covalently, the particular linkage group will depend upon the nature of the two moieties to be bonded. A large number of linking groups and methods for linking are taught in the literature. Broadly, the labels may be divided into the following categories: chromogens; catalyzed reactions; chemiluminescence; radioactive labels; and colloidal-sized colored particles. The chromogens include compounds which absorb light in a distinctive range so that a color may be observed, or emit light when irradiated with light of a particular wavelength or wavelength range, e.g., fluorescers. Both enzymatic and nonenzymatic catalysts may be employed. In choosing an enzyme, there will be many considerations including the stability of the enzyme, whether it is normally present in samples of the type for which the assay is designed, the nature of the substrate, and the effect if any of conjugation on the enzyme's properties. Potentially useful enzyme labels include oxiodoreductases, transferases, hydrolases, lyases, isomerases, ligases, or synthetases. Interrelated enzyme systems may also be used. A chemiluminescent label involves a compound that becomes electronically excited by a chemical reaction and may then emit light that serves as a detectable signal or donates energy to a fluorescent acceptor. Radioactive labels include various radioisotopes found in common use such as the unstable forms of hydrogen, iodine, phosphorus or the like. Colloidal-sized colored particles involve material such as colloidal gold that, in aggregate, form a visually detectable distinctive spot corresponding to the site of a substance to be detected. Additional information on labeling technology is disclosed, for example, in U.S. Pat. No. 4,366,241.

A common method of in vitro labeling of nucleotide probes involves nick translation wherein the unlabeled DNA probe is nicked with an endonuclease to produce free 3′ hydroxyl termini within either strand of the double-stranded fragment. Simultaneously, an exonuclease removes the nucleotide residue from the 5′ phosphoryl side of the nick. The sequence of replacement nucleotides is determined by the sequence of the opposite strand of the duplex. Thus, if labeled nucleotides are supplied, DNA polymerase will fill in the nick with the labeled nucleotides. Using this well-known technique, up to 50% of the molecule can be labeled. For smaller probes, known methods involving 3′ end labeling may be used. Furthermore, there are currently commercially available methods of labeling DNA with fluorescent molecules, catalysts, enzymes, or chemiluminescent materials. Biotin labeling kits are commercially available (Enzo Biochem Inc.) under the trademark Bio-Probe. This type of system permits the probe to be coupled to avidin which in turn is labeled with, for example, a fluorescent molecule, enzyme, antibody, etc. For further disclosure regarding probe construction and technology, see, for example, Sambrook et al. (1989) supra, or Wu et al. (1997) Methods In Gene Biotechnology, CRC Press, New York.

The oligonucleotide selected for hybridizing to the target nucleic acid, whether synthesized chemically or by recombinant DNA methodologies, may be isolated and purified using standard techniques and then preferably labeled (e.g., with 35S or 32P) using standard labeling protocols. A sample containing the target nucleic acid then is run on an electrophoresis gel, the dispersed nucleic acids transferred to a nitrocellulose filter and the labeled oligonucleotide exposed to the filter under stringent hybridizing conditions, e.g., 50% formamide, 5×SSPE, 2× Denhardt's solution, 0.1% SDS at 42° C., as described in Sambrook et al. (1989) supra. The filter may then be washed using 2×SSPE, 0.1% SDS at 68° C., and more preferably using 0.1×SSPE, 0.1% SDS at 68° C. Other useful procedures known in the art include solution hybridization, and dot and slot RNA hybridization. Optionally, the amount of the target nucleic acid present in a sample is then quantitated by measuring the radioactivity of hybridized fragments, using standard procedures known in the art.

Nucleic acid in a sample may also be detected by, for example, a Southern blot analysis by reacting the sample with a labeled hybridization probe, wherein the probe is capable of hybridizing specifically with at least a portion of the target nucleic acid molecule. Nucleic acid in a sample may also be detected by Northern blot analysis. A nucleic acid binding protein may also be used to detect nucleic acid encoding breast cancer-associated proteins.

V. Kits

In one embodiment, the invention provides a kit for detecting a cell-proliferative disorder comprising: (a) a receptacle for receiving a sample; and (b) a first binding moiety which binds specifically to a CRKD marker.

In one embodiment, the invention provides a kit for detecting a cell-proliferative disorder comprising: (a) a receptacle for receiving a sample; (b) a first binding moiety which binds specifically to a CRKD marker; and (c) a reference sample.

In one embodiment, the reference sample may comprise a negative and/or positive control. In that embodiment, the negative control would be indicative of a normal cell type and the positive control would be indicative of cancer. Such a kit may also be used for identifying potential candidate therapeutic agents for treating cancer. In one embodiment, the first binding moiety is labeled. In one embodiment, the kit further comprises a second binding moiety which binds specifically to the first binding moiety.

The above mentioned kit can be used for the detection of any cell-proliferative cancer including, without limitation, breast cancer, cervical cancer, prostate cancer, colon cancer, lung cancer, skin cancer, leukemia, lymphoma, lupus, melanoma or any other type of cancer. In one embodiment the kit is for the detection of breast cancer.

In one embodiment, the binding moiety in the kit is an antibody or fragment thereof which specifically binds to CRKD. Antibodies and binding fragments thereof can be lyophilized or in solution. Additionally, the preparations can contain stabilizers to increase the shelf-life of the kits, e.g., bovine serum albumin (BSA). Wherein the antibodies and antigen binding fragments thereof are lyophilized, the kit can contain further preparations of solutions to reconstitute the preparations. Acceptable solutions are well known in the art, e.g., PBS. In one embodiment, the antibody is a polyclonal antibody, a monoclonal antibody, a humanized antibody, a chimeric antibody, a recombinant antibody, or fragment thereof. In a preferred embodiment, the antibody, or fragment thereof is immunoreactive with the extracellular domain of CRKD or with soluble CRKD.

In other embodiment, the binding moiety in the kit is a peptide which specifically binds to CRKD. Peptide preparations can be lyophilized or in solution. Additionally, the preparations can contain stabilizers to increase the shelf-life of the kits, e.g., bovine serum albumin (BSA). Wherein the peptides are lyophilized, the kit can contain further preparations of solutions to reconstitute the preparations. Acceptable solutions are well known in the art, e.g., PBS.

Kits of the present invention can further include the components for an ELISA assay for measuring CRKD and fragments thereof. Samples to be tested in this application include, for example, blood, serum, plasma, urine, lymph, breast ductal secretions and products thereof.

Alternatively, the kits are used in immunoassays, such as immunohistochemistry to test patient tissue biopsy sections.

The kits may also be used to detect the presence of a CRKD marker in a biological sample obtained from a patient using immunohistocytochemistry.

The compositions of the kit of the present invention can be formulated in single or multiple units for either a single test or multiple tests.

In preferred embodiments, the preparations of the kit are free of pyrogens.

Kits of the present invention can include instructions for the use of the compositions.

VI. Methods of Monitoring Therapy

In one embodiment, the invention comprises a method of monitoring the effectiveness of a treatment for a cell-proliferative disorder in a mammal, comprising quantifying the amount of a CRKD marker in a sample, wherein a decrease in the CRKD marker is indicative of the effectiveness of the treatment. The above-described method can be used to monitor the effectiveness of a cancer treatment. In a preferred embodiment, the method is used to monitor the effectiveness or a breast cancer treatment.

In one embodiment, the concentration of a CRKD polypeptide or fragment thereof is compared to a standard sample obtained from healthy and/or untreated patient. Samples can be collected at discrete intervals during treatment and compared to the standard. It is contemplated that changes in the level of CRKD will be indicative of the efficacy of treatment. It is contemplated that the release of soluble CRKD can be measured in samples such as blood, serum, plasma, urine, lymph, breast ductal secretions and products thereof.

Where the assay is used to monitor progression of a cell-proliferative disorder such as breast cancer or the efficacy of a treatment, the step of detecting the presence and abundance of the marker protein or its transcript in samples of interest is repeated at intervals and these values then are compared, the changes in the detected concentrations reflecting changes in the status of the tissue. For example, an increase in the level of CRKD may correlate with progression of the breast cancer. Where the assay is used to evaluate the efficacy of a therapy, the monitoring steps occur following administration of the therapeutic agent or procedure (e.g., following administration of a chemotherapeutic agent or following radiation treatment). Similarly, a decrease in the level of CRKD may correlate with a regression of the breast cancer.

Thus, breast cancer may be identified by the presence of CRKD as taught herein. Once identified, the breast cancer may be treated using compounds that reduce in vivo the expression and/or biological activity of the CRKD. Furthermore, the methods provided herein can be used to monitor the progression and/or treatment of the disease.

VII. Methods of Treatment

Because CRKD is present at detectably higher levels in breast cancer cells relative to normal breast cells, CRKD may be used as target molecule for cell-proliferative disorders I which CRKD is upregulated. Further, because CRKR is the receptor for CRKD, a skilled artisan may also use CRKR as a target molecule for cell-proliferative disorders I which CRKD is upregulated.

In on embodiment, the invention provides methods and compositions for treating a cell-proliferative disorder. In a preferred embodiment the cell-proliferative disorder is cancer. In a more preferred embodiment, the cancer is breast cancer. In one embodiment, the invention further comprises administering a chemotherapeutic agent.

In another embodiment, the invention provides a method of treating a cell-proliferative disorder in a mammal, comprising administering to the mammal an effective amount of pharmaceutical composition comprising a CRKD antagonist.

In one embodiment, the invention provides a method of treating a cell-proliferative disorder in a mammal, comprising administering to the mammal an effective amount of a compound which binds specifically to a CRKR polypeptide to inactive or reduce the biological activity of CRKR.

In one embodiment, the invention provides a method of treating cancer in a mammal, comprising administering to the mammal an effective amount of the antibody or fragment thereof which binds specifically to a CRKD polypeptide. In one embodiment, the invention provides a method of treating cancer in a mammal, comprising administering to the mammal an effective amount of the antibody or fragment thereof which binds specifically to a CRKR polypeptide. In one embodiment, the antibody or fragment thereof inactivates or reduces the biological activity of the protein.

In one embodiment, the invention provides a method of treating a cell-proliferative disorder in a mammal, comprising administering to the mammal an effective amount of a small molecule, for example, a small organic molecule which inhibits or reduces the biological activity of CRKD.

In one embodiment, the invention provides a method of treating a cell-proliferative disorder in a mammal, comprising administering to the mammal an effective amount of a calcium channel agonist. Calcium channel agonists are well known and may be identified using any method known in the art. See, e.g., U.S. Pat. Nos. 6,653,097 and 5,386,025, which are hereby incorporated by reference. Calcium channel agonists include, but are not limited to, BAYK-8644 and CGP-2392.

In one embodiment, the invention provides a method of treating a cell-proliferative disorder in a mammal, comprising administering to the mammal an effective amount of a compound that modulates the expression of CRKD polypeptide. In one embodiment, the invention provides a method of treating cancer in a mammal, comprising administering to the mammal an effective amount of a compound that modulates the expression of CRKR polypeptide.

A. Anti-Sense Based Therapeutics

In one embodiment, the invention provides a method of modulating a cell-proliferative disorder in a patient comprising modulating the expression of a CRKD polypeptide or a CRKR polypeptide in vivo. In a preferred embodiment the cell-proliferative disorder is cancer. In one embodiment, the cancer is breast cancer. In one embodiment, the modulating of the expression of a CRKD polypeptide or a CRKR polypeptide comprises contacting a cell with a nucleic acid selected from the group consisting of a siRNA probe, an antisense nucleic acid or a ribozyme.

A particularly useful cancer therapeutic envisioned is an oligonucleotide or peptide nucleic acid sequence complementary and capable of hybridizing under physiological conditions to part, or all, of the gene encoding the marker protein or to part, or all, of the transcript encoding the marker protein thereby to reduce or inhibit transcription and/or translation of the marker protein gene. Alternatively, the same technologies may be applied to reduce or inhibit transcription and/or translation of a CRKD polypeptide or a protein which interacts with a CRKD polypeptide such as CRKR.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An -anomeric oligonucleotide forms specific double-stranded hybrids with complementary RNA in which, contrary to the usual -units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related protein such as isoforms or functionally redundant protein. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Ribozyme molecules designed to catalytically cleave an mRNA transcript can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

In addition to administration with conventional carriers, the anti-sense oligonucleotides or peptide nucleic acid sequences may be administered by a variety of specialized oligonucleotide delivery techniques. For example, oligonucleotides may be encapsulated in liposomes, as described in Mannino et al. (1988) BioTechnology 6: 682, and Felgner et al. (1989) Bethesda Res. Lab. Focus 11:21. Lipids useful in producing liposomal formulations include, without limitation, monoglycerides, diglycerides, sulfatides, lysolecithin, phospholipids, saponin, bile acids, and the like. Preparation of such liposomal formulations is within the level of skill in the art (see, for example, in U.S. Pat. Nos. 4,235,871; 4,501,728; 4,837,028; and 4,737,323). The pharmaceutical composition of the invention may further include compounds such as cyclodextrins and the like which enhance delivery of oligonucleotides into cells. When the composition is not administered systemically but, rather, is injected at the site of the target cells, cationic detergents (e.g. Lipofectin) may be added to enhance uptake. In addition, reconstituted virus envelopes have been successfully used to deliver RNA and DNA to cells (see, for example, Arad et al. (1986) Biochem. Biophy. Acta 859: 88-94).

For therapeutic use in vivo, the anti-sense oligonucleotides and/or peptide nucleic acid sequences are administered to the individual in a therapeutically effective amount, for example, an amount sufficient to reduce or inhibit target protein expression in malignant cells. The actual dosage administered may take into account whether the nature of the treatment is prophylactic or therapeutic in nature, the age, weight, health of the patient, the route of administration, the size and nature of the malignancy, as well as other factors. The daily dosage may range from about 0.01 to 1,000 mg per day. Greater or lesser amounts of oligonucleotide or peptide nucleic acid sequences may be administered, as required. As will be appreciated by those skilled in the medical art, particularly the chemotherapeutic art, appropriate dose ranges for in vivo administration would be routine experimentation for a clinician. As a preliminary guideline, effective concentrations for in vitro inhibition of the target molecule may be determined first.

B. Binding Protein-Based Therapeutics

As mentioned above, a cancer marker protein or a protein that interacts with the cancer marker protein may be used as a target for chemotherapy. For example, a binding protein designed to bind the marker protein essentially irreversibly can be provided to the malignant cells, for example, by association with a ligand specific for the cell and known to be absorbed by the cell. Means for targeting molecules to particular cells and cell types are well described in the chemotherapeutic art.

Binding proteins may be obtained and tested using technologies well known in the art. For example, the binding portions of antibodies may be used to advantage. It is contemplated, however, that intact antibodies or BABS that have preferably been humanized may be used in the practice of the invention. As used herein, the term “humanized” is understood to mean a process whereby the framework region sequences of a non-human immunoglobulin variable region are replaced by corresponding human framework sequences. Accordingly, it is contemplated that such humanized binding proteins will elicit a weaker immune response than their unhumanized counterparts. Particularly useful are binding proteins identified with high affinity for the target protein, e.g., greater than about 10⁹ M⁻¹ Alternatively, DNA encoding the binding protein may be provided to the target cell as part of an expressible gene to be expressed within the cell following the procedures used for gene therapy protocols well described in the art. See, e.g., U.S. Pat. No. 4,497,796, and Baichwal, ed. (1986) Gene Transfer. It is anticipated that, once bound by binding protein, the target protein will be inactivated or its biological activity reduced thereby inhibiting or retarding cell division.

As described above, suitable binding proteins for in vivo use may be combined with a suitable pharmaceutically-acceptable carrier, such as physiological saline or other useful carriers well characterized in the medical art. The pharmaceutical compositions may be provided directly to malignant cells, for example, by direct injection, or may be provided systemically, provided the binding protein is associated with means for targeting the protein to target cells. Finally, suitable dose ranges and cell toxicity levels may be assessed using standard dose range experiments. Therapeutically-effective concentrations may range from about 0.01 to about 1,000 mg per day. As described above, actual dosages administered may vary depending, for example, on the nature of the malignancy, the age, weight and health of the individual, as well as other factors.

C. Small Molecule-Based Therapeutics

The skilled artisan can, using methodologies well known in the art, screen small molecule libraries (either peptide or non-peptide based libraries) to identify candidate molecules that reduce or inhibit the biological function of the CRKD. The small molecules preferably accomplish this function by reducing the in vivo expression of the target molecule, or by interacting with the target molecule thereby to inhibit either the biological activity of the target molecule or an interaction between the target molecule and its in vivo binding partner.

It is contemplated that, once the candidate small molecules have been elucidated, the skilled artisan may enhance the efficacy of the small molecule using rational drug design methodologies well known in the art. Alternatively, the skilled artisan may use a variety of computer programs which assist the skilled artisan to develop quantitative structure activity relationships (QSAR) which further to assist the design of additional candidate molecules de novo. Once identified, the small molecules may be produced in commercial quantities and subjected to the appropriate safety and efficacy studies.

It is contemplated that the screening assays may be automated thereby facilitating the screening of a large number of small molecules at the same time. Such automation procedures are within the level of skill in the art of drug screening and, therefore, are not discussed herein. Candidate peptide-based small molecules may be produced by expression of an appropriate nucleic acid sequence in a host cell or using synthetic organic chemistries. Similarly, non-peptidyl-based small molecules may be produced using conventional synthetic organic chemistries well known in the art.

As described above, for in vivo use, the identified small molecules may be combined with a suitable pharmaceutically acceptable carrier, such as physiological saline or other useful carriers well characterized in the medical art. The pharmaceutical compositions may be provided directly to malignant cells, for example, by direct injection, or may be provided systemically, provided the binding protein is associated with means for targeting the protein to target cells. Finally, suitable dose ranges and cell toxicity levels may be assessed using standard dose range experiments. As described above, actual dosages administered may vary depending, for example, on the nature of the malignancy, the age, weight and health of the individual, as well as other factors.

D. Pharmaceutical Compositions

One embodiment of the present invention are methods of treating a cell-proliferative disorder, preferably cancer, more preferably breast cancer, with pharmaceutical compositions of antibodies, antigen binding fragments, peptides and compounds as described above. In a preferred embodiment, the patient receiving treatment is a human patient. Pharmaceutical compositions of the antibodies, antigen binding fragments, and peptides can be administered to a patient in need there of by injection.

Pharmaceutical compositions of the present invention are administered in a therapeutically effective amount which are effective for producing some desired therapeutic effect by inducing tumor-specific killing of tumor cells in a patient and thereby blocking the biological consequences of that pathway in the treated cells eliminating the tumor cell or preventing it from proliferating, at a reasonable benefit/risk ratio applicable to any medical treatment.

In one embodiment of the present invention, the pharmaceutical compositions are formulated to be free of pyrogens such that they are acceptable for administration to human patients. Testing pharmaceutical compositions for pyrogens and preparing pharmaceutical compositions free of pyrogens are well understood to one of ordinary skill in the art.

One embodiment of the present invention contemplates the use of any of the pharmaceutical compositions of the present invention to make a medicament for treating cancer. Medicaments can be formulated based on the physical characteristics of the patient/subject needing treatment, and can be formulated in single or multiple formulations based on the stage of the cancerous tissue. Medicaments of the present invention can be packaged in a suitable pharmaceutical package with appropriate labels for the distribution to hospitals and clinics wherein the label is for the indication of treating a specific cancer in a subject. Medicaments can be packaged as a single or multiple units. Instructions for the dosage and administration of the pharmaceutical compositions of the present invention can be included with the pharmaceutical packages.

In one preferred embodiment, pharmaceutical compositions of the present invention can be administered to a patient by any convenient route, including, for example, subcutaneous, intradermal, intravenous, intra-arterial, intraperitoneal, or intramuscular injection.

E. Combination Therapy

In a preferred embodiment, the antibodies, antigen binding fragments, or peptides are labeled with a radiolabel or a toxin that kills the target cell upon binding of the antibodies, antigen binding fragments, or peptides to CRKD.

In one embodiment of the present methods, the toxin is any one of ricin, ricin A chain (ricin toxin), Pseudomonas exotoxin (PE), diphtheria toxin (DT), Clostridium perfringens phospholipase C (PLC), bovine pancreatic ribonuclease (PBR), pokeweed antiviral protein (PAP), abrin, abrin A chain (abrin toxin), cobra venum factor (CVF), gelonin (GEL), saporin (SAP) modeccin, viscumin or volkensin.

In one embodiment of the present methods, the radiolabel is any one of the following radionuclides: ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81M}Kr, ^{87M}Sr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶H ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Preferred therapeutic radionuclides include ¹⁸⁸Re, ¹⁸⁶Re, ²⁰³Pb, ²¹²Pb, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Au, ¹⁶⁶Ho, or ¹⁷⁷Lu.

Subject antibodies, antigen binding fragments, peptides and peptidomimetics of the present invention can also be used in combination therapy with chemotherapeutic agents such as the chemotherapeutic agents discussed above.

The pharmaceutical compositions can be administered separately or concomitantly. In one aspect of the present invention, the pharmaceutical compositions are administered in a single formulation. In one aspect of the present invention, the pharmaceutical compositions are administered as separate formulations.

VIII. Drug Screening Assays

The invention also comprises methods to screen for compounds which can be used to treat a cell-proliferative disorder such as cancer.

In one embodiment, the method comprises (a) identifying a CRKD antagonist, and (b) determining whether said CRKD antagonist is effective against a cell-proliferative disorder. Said methods can be carried out using methods which are well known in the art. For example, determining whether a CRKD antagonist is effective against a cell-proliferative disorder can be carried out using any in vitro or in vivo models of a cell-proliferative disorder.

The invention also comprises a method to screen for CRKD antagonists, comprising: (a) contacting a CRKD polypeptide with a test compound under conditions suitable for detecting the binding of the CRKD polypeptide to the test compound, (b) determining whether the test compound binds the CRKD polypeptide, and (c) further determining whether the test compound prevents, inhibits or reduces the binding of CRKD to CRKR, wherein a test compound that binds the CRKD polypeptide and prevents, inhibits or reduces inhibits the binding of CRKD to CRKR is a CRKD antagonist. In one embodiment the method further comprises determining whether the test compound binds the extracellular domain of said CRKD polypeptide.

IX. Methods of Conducting a Business

The invention further comprises a method of conducting a business comprising: (a) obtaining a sample; (b) detecting the presence of a CRKD marker in the sample; and (c) reporting the results of such detection. In one embodiment, the method further comprises quantifying the amount of the CRKD marker in the sample. The sample may be obtained from any individual, including without limitation a patient or a health care provider. The sample may be any biological sample described in the instant application. The CRKD marker may be detected or quantified using any of the methods described in the instant application. The method can be used to conduct a diagnostic business.

The invention also comprises a method of developing a business comprising: (a) identifying one or more CRKD antagonists; (b) generating a composition comprising said CRKD antagonist; (c) conducting therapeutic profiling of said composition for efficacy and toxicity; (d) preparing a package insert describing the use of said composition; and (d) marketing said composition. In one embodiment, the composition is used to treat a cell-proliferative disorder.

The invention also comprises a method of developing a business comprising: (a) identifying one or more CRKD antagonists; (b) generating a composition comprising a said CRKD antagonist, wherein said composition can be used to treat a cell-proliferative disorder, and (c) licensing, jointly developing or selling, to a third party, the rights for selling the composition.

X. Microarrays

In one embodiment, the invention comprises a microarray comprising at least one or more probes for detecting a CRKD marker. In one embodiment, the microarray further comprises one or more probes for detecting a CRKR marker. In one embodiment, the microarray is used to detect or quantify a CRKD marker.

In a preferred embodiment, the microarray is used to asses the CRKD status of a patient. In another embodiment, the microarray is used to diagnose or augment the diagnosis of a cell-proliferative disorder such as cancer.

As used herein, an “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

A “nucleic acid library array” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports).

As used herein, the term “array” is meant to include those libraries of nucleic acids which can be prepared by spotting nucleic acids of essentially any length (e.g., from 1 to about 1000 nucleotide monomers in length) onto a substrate. The term “substrate” refers to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods have been shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, NY, 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Patent application Ser. Nos. 09/916,135, 09/920,491, 09/910,292, and 10/013,598. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S., 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

XI. Methods of Identifying Mammary Stem Cells

In one embodiment CRKD is used a mammary stem cell marker which can be used to identify and isolate stem cells.

In one embodiment the invention provides a method to identify the presence of mammary stem cells in a mixed cell population, comprising detecting the presence of a CRKD marker, wherein the presence of CRKD polypeptide is indicative of the presence of mammary stem cells in a mixed cell population.

In another embodiment the invention provides a method for isolating mammary stem cells comprising: (a) obtained a mixed cell population; (b) exposing said mixed cell population to a binding moiety specific for and a CRKD marker; and (c) separating the cells bound to the binding moiety, thereby isolating mammary stem cells.

The CRKD marker can be any of the CRKD markers described above. In one embodiment, the CRKD marker is a CRKD polypeptide or a fragment thereof. In another embodiment, the CRKD marker is a nucleic acid encoding a CRKD polypeptide. In one embodiment, the nucleic acid is an mRNA molecule.

The expression of the CRKD marker can be determined at the mRNA or protein level using any suitable assay system. In one embodiment, the presence of the CRKD marker is detected using a binding moiety. In one embodiment, the binding moiety is an antibody or a fragment thereof. In another embodiment, the presence of the CRKD marker is detected using PCR amplification, fluorescence labeling, or immunocytochemistry.

In another embodiment, the invention comprises a method for isolating mammary stem cells comprising: (a) obtained a mixed cell population; (b) exposing said mixed cell population to a binding moiety specific for a CRKD marker; and (c) separating the cells bound to the binding moiety, thereby isolating mammary stem cells.

IX. Equivalents

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### The Identification of a Novel Calcineurin-Regulated Gene by Microarray Analysis

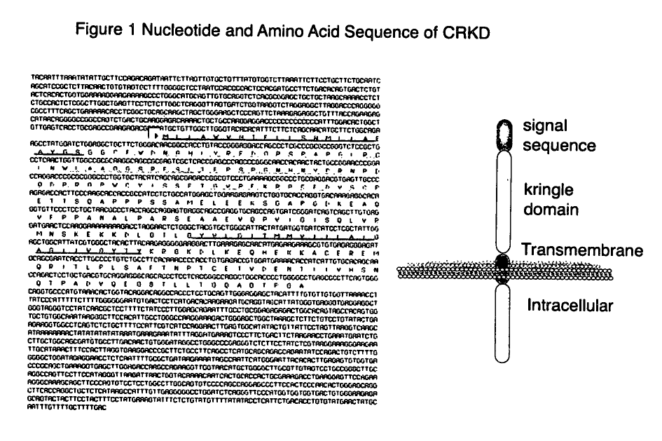

Classic immunological studies have identified several cytokines as targets of calcineurin/NFAT (1, 37), but little is known of the genes controlled by this pathway during development. Therefore, we compared the expression profiles of E9.5 whole calcineurin B-null [CnB^{*/*}, (12)] and wild-type embryos. 23 somite embryos were used since we obtained highly variable results when using embryos aged by vaginal plug only (data not shown). This mid-gestational stage was used as CnB^{*/*} embryos are clearly affected but still alive at this time (12). Four independent collections of RNA from CnB^{*/*} and wild-type littermate embryos were used and hybridized to oligonucleotide arrays representing ˜36,000 transcripts. Data from the four experimental (CnB^{*/*}) arrays were then intercompared to the four standard (wild-type) arrays to give a final query set of sixteen. Transcripts increased or decreased by at least 2.5-fold in the CnB^{*/*} embryos in all sixteen queries were considered for futher analysis. Given the pleiotropic effects of calcineurin/NFAT, we reasoned that our targets would encode secreted and/or transmembrane proteins. To this end, of the transcripts that met our criteria, we cloned only those genes with putative hydrophobic signal sequences (13). A search of several public and private databases revealed that one target, the HGFL gene (GenBank Accession AF528081), was represented only in vertebrate genomes, indicative of the targets we sought. This 2.5-kb transcript encodes a 264 amino acid protein (FIG. 1A) that was found to be increased in the CnB^{*/*} embryos by an average of 3.7-fold. The encoded protein contains a putative signal peptide and a single kringle domain, regions known to be important in a variety of developmental and pathological processes (18, 19). A second hydrophobic stretch downstream of the kringle domain suggests that it is a type I transmembrane protein (FIG. 1B).

### Example 2

### CRKD is a Calcineurin-Repressed Transmembrane Protein

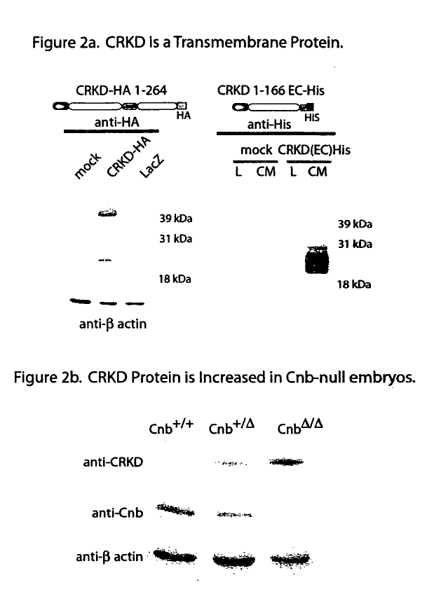

In order to determine if the signal peptide is functional, the putative extracellular and full-length sequences were expressed in 293T cells by transient transfection. As shown in FIG. 2a, the extracellular portion (EC) is efficiently secreted while the full-length-remains within the cell, demonstrating that the protein is indeed transmembrane. In order to verify the arrays, we raised polyclonal antibodies to the extracellular domain and performed Western blot analysis comparing Cnb wild-type, heterozygotic (loxP/Δ), and homozygotic-null (Δ/Δ) (37) E9.5 embryos. As demonstrated in FIG. 2b protein levels of this target are dramatically increased in the null embryos, and are even slightly elevated in the heterozygous embryos. Therefore, we have named this the calcineurin-regulated kringle domain (CRKD) gene.

### Example 3

### CRKD is Specifically Expressed in Undifferentiated Mammary Epithelium

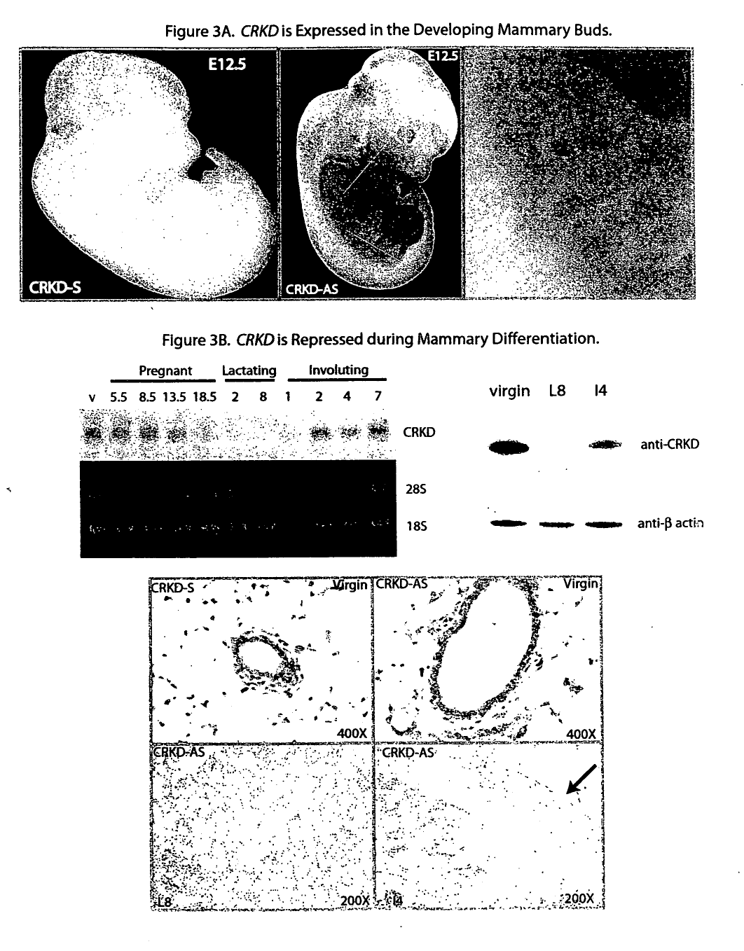

In order to define a role for CRKD in development, whole-mount in situ hybridization was done on E12.5 embryos. One of the most striking areas of expression is seen as ‘dots’ along the lateral ridge between the fore and hind limbs (FIG. 3A). These structures are the condensed epithelium of the mammary buds, which are surrounded by a ring of stromal tissue not marked by CRKD (20). Given the dynamic nature of the mammary gland (21), we determined the developmental regulation of CRKD in this tissue by Northern blot. As shown in FIG. 3B, CRKD is expressed in the virgin mammary gland as well as tissue from early and mid-gestational (up to P13.5) glands. In contrast, CRKD is completely repressed during late-stage pregnancy and lactation, the only periods of functional differentiation in the mammary gland (21). CRKD expression is then de-repressed at the second day of involution (12) while the mammary gland is undergoing remodeling. This demonstrates that CRKD is only expressed in the undifferentiated mammary gland. The same pattern of expression is seen at the protein level (FIG. 3B). Given the enormous increase of mammary tissue mass during lactation, we were concerned that CRKD levels were simply being diluted instead of repressed. Therefore, we examined CRKD levels in paraffin sections by in situ hybridization. As shown in FIG. 3B, CRKD is expressed in the ductal epithelium of the virgin gland. In contrast, CRKD is not detectable in the lactating (L8) gland. Finally, in accordance with the Northern blot results, the involuting (14) gland displays a de-repression of CRKD expression.

### Example 4

### CRKD is Specifically Shed from Mammary Cancer Cell Lines and is Found in the Serum of Breast Cancer Patients

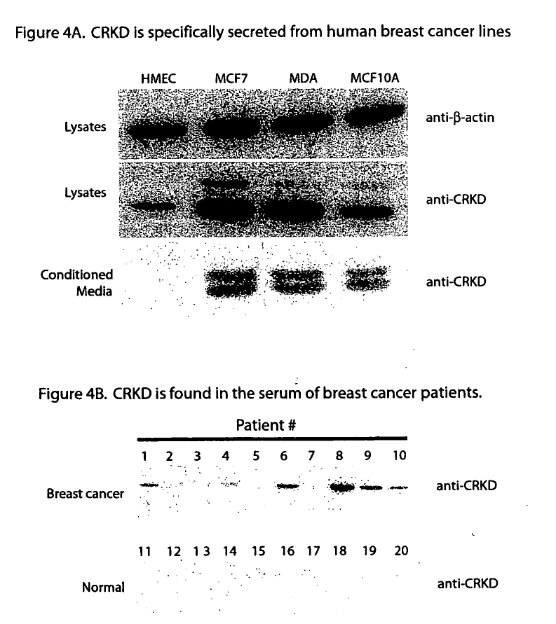

Since we found distinct CRKD expression specifically in the undifferentiated mammary gland, we searched public databases to see if there is any correlation between CRKD expression and breast cancer. Perou et al. (22) have published a comprehensive microarray study of breast cancer, and the full findings are published on the Stanford Microarray Web site (http ://genome-www5.stanford.edu/cgibin/SMD/publication/viewPublication_pl?pubno=38). A search of their data found ESTs representing CRKD were over-expressed in all samples from breast cancer patients. Given this, we assayed CRKD expression in breast cancer cell lines. As shown in FIG. 4A, CRKD protein levels are dramatically elevated in three breast cancer cell lines (MCF7; MDA-MB-231, and the immortalized MCF10A line) as compared to primary human mammary epithelial cells (HMEC). Furthermore, we were surprised to find that CRKD was specifically shed into the media of the breast cancer cell lines and not the HMEC cultures. These data suggest that CRKD may play a role or at least may serve as a marker for breast cancer.

To this end, we obtained sera from ten individual women with metastatic breast cancer and ten women with no history of disease. One milliliter of the serum was immunoprecipitated with affinity-purified anti-CRKD and the presence of CRKD was subsequently detected by Western blot analysis. FIG. 4B shows that CRKD was indeed detected in seven out of ten (Patient # 1, 2, 4, 6, 8, 9, 10) samples from the patients with metastatic breast cancer, while no CRKD was detected in the normal serum. These results suggest that CRKD is a potential serum marker for breast cancer.

### Example 5

### Expression Cloning of a Putative CRKD Binding Partner

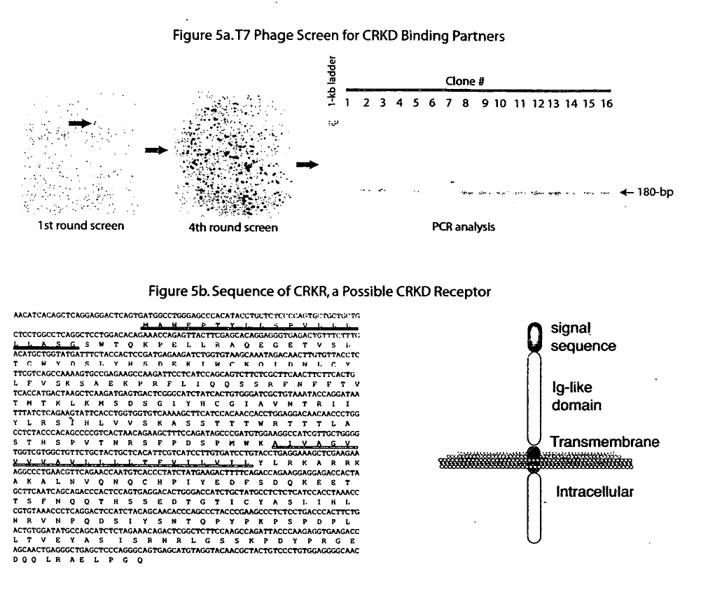

Given that we found CRKD to be both a transmembrane and secreted protein, we reasoned that it might bind to a receptor. To this end we performed a biopanning screen for CRKD binding partners using a T7 phage human breast cancer library and CRKD(EC)His as bait. Following four rounds of screening, amplified phage were plated-out and assessed for binding by ‘Far Western’ (17). Greater than 95% of phage tested positive for CRKD(EC)His binding (FIG. 5A), while fewer than 5% of the phage from the BSA negative control screen demonstrated the same characteristic (data not shown). Sixteen phage from the CRKD(EC)His were randomly picked and screened by PCR and direct DNA sequencing. Fourteen of the sixteen phage amplified the same 180 by insert (FIG. 5A), all of which were sequenced. A portion of the resulting cloned cDNA is shown in FIG. 5B (the entire cDNA can be found on GenBank, Accession AY522648), and represents a novel gene with a predicted immunoglobulin (Ig)-like region, hence we have named this the CRKD receptor (CRKR). CRKR is identical to RIKEN cDNA B430306N03 and encodes a protein of 289 amino acids in length and is probably transmembrane. Given the very stringent binding conditions used and the fact that 87.5% of the represented clones from the screen were CRKR, it is quite likely that this interaction is real, although more definitive studies will be required.

Discussion

We describe here the cloning of CRKD from a genomic screen for calcineurin/NFATregulated genes involved in vertebrate development. CRKD was over-expressed in Cnb-null embryos and encodes a transmembrane protein with a predicted kringle domain. Our analysis found CRKD to be a marker for embryonic mammary development and expressed specifically in the undifferentiated adult gland. In addition, CRKD is over-expressed in breast cancer and the extracellular domain is found in the serum from breast cancer patients. Finally, we report the cloning of CRKR, itself a predicted transmembrane protein that may serve as a receptor for soluble CRKD or a binding partner in hetero- or homotypic cellular interactions. Taken together, these data suggest a previously unappreciated role for calcineurin/NFAT in mammary gland development, identify a potential serum marker for breast cancer, and define a putative receptor for CRKD.

The mammary gland is a well-studied organ that undergoes characteristic morphological and genotypic changes throughout development (21). In the mouse, mammary gland formation begins around day 10 of gestation on the surface ectoderm of both lateral flanks of the embryo (23). By E11.5 five bilateral, paired thickenings of the ectoderm appear known as the mammary placodes, which will develop into bud-like structures that are located at precise points along the antero-posterior axis of the murine embryo, and are surrounded by a ring of mesenchymal tissue (24). Some genes have been identified as markers of embryonic mammary epithelial and stromal development, including the transcription factors Lefl (24) and Hoxb9 (20), respectively. Given its expression in the developing mammary epithelium by E12.5, and that it is a transmembrane protein, CRKD should prove important in the delineation of signaling pathways mediating mammary development.

In the adult, virgin mice have glands that are quiescent and undifferentiated. During pregnancy, the glands rapidly proliferate and begin to differentiate in the later stages (around day 15 of gestation), preparing for subsequent lactation. Finally, upon forced or natural weaning, the mammary gland undergoes massive apoptosis and matrix remodeling to prepare for subsequent pregnancies [see (21) for review]. Several genes are used for markers of mammary differentiation, including ZNF143 (25), StatS, casein β, and whey acidic protein (26).

Conversely, little is known about genes specific for the undifferentiated gland. Recent work has concentrated on the so-called cap cells, the multipotent progenitor population of the mammary gland of which P-cadherin is a marker (27). These cells are found in the apical leading edge of the terminal end bud, the functional unit of the mammary gland, and have been proposed to serve as the mammary stem cell population and play a role in oncogenesis of the breast (28). Given the essential role of this population in mammary gland development and possibly tumorigenesis, it is essential to identify the molecules that regulate cap cell growth, survival, and differentiation. To this end, recently published work analyzed a putative mammary stem cell population by microarray analysis in order to define molecular markers of these undifferentiated progenitors (29). Interestingly, one of the genes identified as ‘expressed in differentiated cells only’ was Cnb (protein phosphatase 3). This is consistent with our findings that CRKD, which is found only in the undifferentiated gland, is negatively regulated by Cnb. Since CRKD is a transmembrane protein, it is possible that it may be a useful cell surface marker to facilitate purification of mammary stem cells in order to study not only development but tumorigenesis as well.

Breast cancer is one of the most common cancers and the second leading cause of cancer mortality in women, with approximately one in nine women being affected in their lifetime (30, 31). Hereditary breast cancer, such as those with BRCA-1 and BRCA-2 mutations, account for only 5-10% of all breast cancers (32). Therefore, it is imperative to delineate molecular factors responsible for the development of sporadic breast cancers. Most importantly, a reliable detection marker for breast cancer would allow more effective early treatment. Much attention has been given to this need, mostly focused on large proteomic and genomic studies to identify differentially expressed genes (33, 22). In at least one study, ESTs representing CRKD were found to be over-expressed in all cancers analyzed (22). In accordance, we found CRKD levels to be elevated in and specifically shed from breast cancer cell lines. Given that we have been unable to find a transcript for a secreted form of CRKD, it is conceivable that the CRKD found in the conditioned medium of breast cancer lines is a result of ‘shedding’, a process known to play a major role in mammary development and tumorigenesis (34). We found CRKD present in the serum of 7 out of 10 samples from breast cancer patients, suggesting that it may serve as an early detection marker. Although this is a very small sample size and focused solely on metastatic disease, it is encouraging that CRKD can be detected using only one milliliter of serum and the relatively insensitive method of immunoprecipitation and Western blot. Ultimately, a high-throughput ELISA or similar capacity test should be developed.

Several therapeutic drugs have been developed towards secreted proteins and intercellular signaling networks [see (35) for review], and their clinical success suggests that this approach is valid and new targets need to be found. To this end, the discovery of novel extracellular signaling molecules has intensified and led to the development of consortiums conducting large screens (36). We have identified a novel transmembrane protein, CRKD, and its putative binding partner CRKR, and find that CRKD is over-expressed in breast cancer lines and present in the serum of breast cancer patients. Interestingly, ESTs representing CRKR are found in bone and axillary lymph nodes, primary areas of breast cancer metastasis (38). In addition, CRKR ESTs are represented in adipose tissue, which surrounds the breast epithelium where CRKD is expressed. Therefore, it is tempting to speculate that CRKR and CRKD serve as homing molecules for heterotypic cellular interactions. In addition, CRKD is found only in undifferentiated mammary tissue, and its negative regulator, Cnb, has been reported to be expressed exclusively in differentiated mammospheres (29). Therefore, CRKD may be an accessible target during the process of mammary stem cell transformation and therapies derived towards CRKD may help combat breast cancer.

Experimental Methods Used in the Examples Described Above:

Microarray Analysis.

Four 23-somite CnB^{*/*} (12) and wild-type littermate embryos were collected and RNA extracted (Totally RNA kit, Ambion). T7-(dT)₂₄,-primed double stranded cDNA was then produced employing the SuperScript II kit (Invitrogen) using 10 μg of total RNA as template, followed by three phenol-chloroform extractions and ethanol precipitation. Biotin-labeled cRNA was then produced (Enzo BioArray kit, Affymetrix) and purified (RNeasy system, Qiagen). The biotinylated cRNA was fragmented at 94° C. for 35 minutes with 0.2 M Tris-acetate [pH 8.1], 150 mM MgOAc, and 500 mM KOAc. The fragmented cRNA was hybridized to the U74v2 series (A, B, and C ‘chips’) oligonucleotide arrays (Affymetrix) by the Stanford Microarray Facility. This procedure was repeated independently four times. The four experimental (CnB^{*/*}) array were then intercompared to each standard (wild-type) array, for a total of 16 comparisons. Data were analyzed using MicroArray Suite 5.0 and the Data Mining Tool (Affymetrix). Transcripts that were changed at least 2.5-fold in the CnB^{*/*} samples for all 16 comparisons were used for further analysis.

Cloning of CRKD.

Of the transcripts that met our criteria, 47 were considered novel or ESTs. These transcripts were cloned and then scanned for hydrophobic signal sequences (13) to find secreted and/or transmembrane proteins. One such transcript, AI846040, was identified and further analyzed. In order to clone this particular transcript, the EST AI846040 was ordered (I.M.A.G.E. consortium) and used as a probe to screen 1.2×10⁶ recombinants from an E10.5 cDNA library using standard techniques. Nine independent clones were carried through four rounds of screening and assembled to produce the 2539-kb transcript (GenBank Accession AY522649).

Expression Vector and Riboprobe Construction.

The pCRKD/HA plasmid was constructed by PCR and TOPO cloning into the pcDNA3.1V5-His-TOPO vector (Invitrogen) using one of the identified clones from the library screen as template with the primers: F 5′ CACCATGCTGTTGGCTTGG 3′ and R 5′ TCAAGCGTAGTCTGGAACGTCATATGGGTAGGCCCAGGGGTGCC3′. The pCRKD(EC)/His vector was constructed in a similar manner using the same forward primer and the reverse primer 5′ TCAATGGTGATGGTGATGATGGTCTTTTTTTTCCTTGGAG 3′ and produces a six-His-tagged extracellular domain (amino acids 1-166) of CRKD. The bacterial-expression plasmid pGST-CRKD(EC) was constructed by PCR into the pGEX-2T vector (Amersham Biosciences). The pcDNA-LacZ-V5/His was from Invitrogen. All clones were verified by sequencing in both directions. Sense and antisense digoxigenin-labeled riboprobes were produced using the plasmids p126-CRKD-5′ and p492-CRKD-3′ and the T7 and T3 RNA polymerases (Boehringer Mannheim) as per the maufacturer's protocol. The resulting riboprobes hybridize to the 5′ and 3′ UTRs of CRKD, respectively.

Northern Blot Analysis.

The mammary aging blot was purchased from Seegene (Seoul, S. Korea) and contains 10 μg of total RNA per lane. The [³²P]dCTP-labeled probe was produced by random primer labeling using the p492-CRKD-3′ cDNA as template. Northern blot analysis was carried out using ExpressHyb (Clontech) according to the manufacturer's instructions.

Whole-Mount and Tissue Section in Situ Analysis.

All solutions were DEPC-treated and autoclaved prior to use. For whole-mount analysis, E12.5 CD1 embryos were collected and fixed in 4% paraformaldehyde in phosphate-buffered saline [pH 7.4]/0.1% Tween20 (PBST) overnight at 4° C., followed by dehydration through a methanol-PBST series of 0%, 25%, 50%, 75%, and 100% for five minutes each at room temperature, and then stored in 100% methanol at −20° C. until use. Embryos were re-hydrated through a graded methanol-PBST series of 75%, 50%, 25% and 0% for five minutes each at room temperature, followed by treatment with 6% hydrogen peroxide in PBST for 1 hour at room temperature. 10 μg/ml Proteinase K in PBST was added for 25 minutes at room temperature and quenched with 2 mg/ml glycine. Embryos were re-fixed in 4% paraformaldehyde-0.2% glutaraldehyde for 20 minutes at room temperature, then pre-hybridized in 50% formamide, 2×SSC [pH 5.0], 1% SDS, 50 μg/ml heparin, and 50 μg/ml yeast tRNA for one hour at 65° C. Embryos were then hybridized with 1 μg/ml digoxigenin-labeled sense or antisense ribroprobe in pre-hybridization buffer overnight at 65° C. Embryos were washed three times with 50% formamide, 2×SSC [pH 5.0], 1% SDS for 30 minutes at 70° C., three times with 50% formamide, 2×SSC [pH 5.0] for 30 minutes at 65° C., and then three times with PBST for five minutes at room temperature. Embryos were blocked in 10% sheep serum-PBST for 2.5 hours at room temperature, and then incubated with 1:3000 embryo powder-subtracted anti-digoxigenin-alkaline phosphatase (Roche) overnight at 4° C. Following six washes far one hour each with PBST at room temperature, embryos were incubated with AP buffer (100 mM Tris-HCl [pH 9.5], 50 mM MgCl₂, 100 mM NaCl, 0.1% Tween20, and 2 mM levamisole) twice for 20 minutes at room temperature. Finally, embryos were developed with BM Purple (Roche) at room temperature' in the dark.

The #4 mammary glands were collected, stretched on slides and fixed, dehydrated, and re-hydrated as above. Glands were then washed twice with 100% ethanol for 20 minutes and twice with xylene for 20 minutes. Melted paraffin wax was then added for one hour at 55° C., and then glands were incubated with fresh wax overnight at 55° C. Glands were then washed once with fresh wax at incubated for one hour at 55° C., and sections cut at 5 μM. In situ hybridization was then carried out as using a tyramide amplification protocol as described (14).

Antibody Production.

15 L of E. coli strain BL21 harboring the pGST-CRKD(EC) plasmid was grown in LB plus 50 ug/ml ampicillin at 37° C. until an OD₆₀₀ of 1.0. IPTG was then added to 0.5 mM and incubation was carried out for 5 further hours. Bacterial pellets were spun down, sonicated, and then incubated in % Triton X-100 for 30 minutes at room temperature. Insoluble material was then pelleted and soluble GST-CRKD(EC) purified with glutathione sepharose 4B (Amersham Biosciences). This resulted in a consistent yield of ˜200 μg/L of GST-CRKD(EC), as only ˜5% of the CRKD was soluble in bacteria. 2.5 mg of purified GST-CRKD(EC) was used to produce rabbit polyclonal antibodies (Covance). For affinity purification, recombinant CRKD(EC)/His (6-His-tagged extracellular domain, amino acids 1-166) was produced by transfection of 10⁸ 293T cells with pCRKD(EC)/His using LipofectAMINE 2000 (Invitrogen). 24 hour post-transfection the medium was changed to OPTI-MEM (Gibco) and incubated for 4 days. The conditioned medium (75 ml) was collected and filtered through a 0.45 μm membrane into 20 mM HEPES [pH 7.4], 0.05% sodium azide, 300 mM NaCl, 20 mM imidazole, and 0.5% Protease Inhibitor Cocktail III (Calbiochem). The pH of the final solution was adjusted to 8.0 with NaOH, and Ni²⁴-NTA agarose (Qiagen) was added and incubated for two hours at 4° C. The beads were washed four times with 50 mM NaH₂PO₄ [pH 8.0], 300 mM NaCl, 20 mM imidazole, and 0.05% Tween20. Purified CRKD(EC)His (˜2 mg) was eluted with 500 mM imidazole [pH 6.0], microdialyzed (Pierce) into PBS with 0.5% Protease Inhibitor Cocktail III (Calbiochem), and added to swollen CNBr-activated sepharose 4B (Amersham Biosciences) to form the affinity column. Affinity purification of anti-CRKD antibodies was carried out as described (15).

Transfection, Western Blot and Immunoprecipitation (IP).

Where indicated, cells were transfected using LipofectAMINE 2000 (Invitrogen) in OPTI-MEM (Gibco) as per the manufacturer's protocol. Primary human mammary epithelial cells (HMEC) were from Cambrex, while MCF7, MDA-MB-231, and MCF10A cells were from ATCC. All mammary cells were grown in fully-supplemented, serum-free MEGM (Cambrex). Cells were washed twice with PBS and lysed in RIPA (50 mM Tris-HCl [pH 7.4], 1% NP-40, 0.25% Na-deoxycholate, 0.1% SDS, 150 mM NaCl, 1 mM EDTA, 1 mM Na₃VO₄, 1 mM NaF, 0.5% Protease Inhibitor Cocktail III), transferred to microfuge tubes and incubated on ice for thirty minutes. Insoluble material was then pelleted (13,000 rpm for ten minutes at 4° C.) and protein concentrations determined by Bradford assay (BioRad). For conditioned OPTI-MEM medium, 0.5% Protease Inhibitor Cocktail III was added to one milliliter and concentrated to ˜50 μl using Microcon 3000 MWCO (Millipore). E9.5 embryo propers were homogenized in RIPA by tituration and incubated on ice for thirty minutes, and protein concentrations were determined by Bradford assay. Yolk sacs were reserved and genotyped as described (37). The #4 mammary gland was dissected and homogenized in 2 ml RIPA and incubated on a rocker at 4° C. for one hour. Insoluble material was spun down and lysates, minus fat, were collected and protein concentrations determined by Bradford assay. Equal amounts of protein (20 μg) were brought to equal volume with RIPA, solubilized by the addition of 6× loading dye at 95° C. for five minutes, and separated by 12.5% SDS-PAGE. Proteins were transferred to nitrocellulose (Millipore), placed in block buffer (5% milk in Tris-buffered saline/Tween [10 mM Tris-HCl, pH 8.0, 1 M NaCl, 0.1% Tween20], TBST) for one hour at room temperature, and incubated overnight with primary antibody in block buffer at 4° C. Membranes were extensively washed and incubated with secondary horseradish peroxidase (HRP)-coupled antibodies, followed by detection with ECL Plus (Amersham Biosciences). Antibodies used were anti-HA 3F10 (Roche, 1:1000), anti-His (Santa Cruz, 1:500), anti-CRKD (1:2000), anti-β actin (Sigma, 1:5000), and anti-Cnb (Sigma, 1:3000). Where indicated membranes were stripped and re-probed as described (16).

For IPs, affinity-purified anti-CRKD was cross-linked to Protein A-sepharose (Amersham Biosciences) and 10 μl of coupled beads was added to one milliliter of freshly-obtained, Protein A-depleted serum with 0.5% Protease Inhibitor Cocktail III added. The IP was carried out overnight at 4° C. followed by extensive washing with TBST, and bound antigen was released by addition of 100 mM glycine, pH 2.5. Eluted protein was then analyzed by Western blot as described above.

Biopanning Screen and CRKR Cloning.

The T7Select Human Breast cDNA library (Novagen) was used for a biopan screen according to the manufacturer's and published protocols (17). Briefly, an ELISA plate was coated with 1 μg/ml purified CRKD(EC)His or BSA (negative control) and used to screen 10⁹ clones from the phage library. Phage were allowed to bind for one hour at room temperature, extensively washed, and eluted with 1% SDS. Bound phage were then amplified in BLT5615 E. coli and used for three more rounds of screening. Following the fourth and final round of screening, bound phage were amplified and plated onto 0.6% top agarose LB plates and transferred to nitrocellulose. The membranes were then blocked with 5% milk and incubated with 0.5 μg/ml CRKD(EC)His overnight at 4° C. Following extensive washing, bound bait proteins were detected by anti-His antibodies and chemiluminescence (Amersham). Sixteen positive-binding phage were picked and used to amplify and sequence inserts by PCR using the T7SelectUP and T7SelectDOWN primers (Novagen). The insert that represented 14/16 clones was then used as a probe to screen 1.2×10⁶ recombinants from an E10.5 cDNA library using standard techniques. Ten independent clones were carried through four rounds of screening and assembled to produce the 3465-bp transcript (GenBank Accession AY522648).

Abbreviations:

NFAT, nuclear factor of activated T cells; CRKD, calcineurin-regulated kringle domain; CRKR, CRKD receptor; NFATc, cytoplasmic NFAT; NMDA, N-methyl-d-aspartate; CRAG, Ca(2+)-release-activated Ca(2+); EST, expressed sequence tag; CnB, calcineurin B.

Sequences:

SEQ ID NO:1 (GenBank Accession No. AY522649)
SEQ ID NO:2 (GenBank Accession No. AAS13454)
SEQ ID NO:3 (GenBank Accession No. NM_{—}052880)
SEQ ID NO:4 (GenBank Accession No. NP_{—}443112)
SEQ ID NO:5 (GenBank Accession No. AY522648
SEQ ID NO:6 (GenBank Accession No. AAS13453)

### REFERENCES

1. Shaw, J. P., Utz, P. J., Durand, D. B., Toole, J. J., Emmel, E. A., and Crabtree, G. R. (1988) Science 241, 202-205
2. Emmel, E. A., Verweij, C. L., Durand, D. B., Higgins, K. M., Lacy, E., and Crabtree, G. R. (1989) Science 246, 1617-1620
3. Flanagan, W. M., Corthesy, B., Bram, R. J., and Crabtree, G. R. (1991) Nature 352, 803-807
4. Clipstone, N. A., and Crabtree, G. R. (1992) Nature 357, 695-697
5. Graef, I. A., Gastier, J. M., Fraucke, U., and Crabtree, G. R. (2001) Proc Natl Acad Sci USA 98, 5740-5745
6. Graef, I. A., Chen, F., and Crabtree, G. R. (2001) Carr Opin Genet Dev 11, 505-512
7. Graef, I. A., Wang, F., Charron, F'., Chen, L., Neilson, J., Tessier-Lavigne, M., and Crabtree, G. R. (2003) Cell 113, 657-670
7. Crabtree, G. R. (1989) Science 243, 355-361
9. Northrop, J. P., Ho, S. N., Chen, L., Thomas, D. J., Timmerman, L. A., Nolan, G. P., Admon, A., and Crabtree, G. R. (1994) Nature 369, 497-502
10. Park, S., Uesugi, M., and Verdine, G. L. (2000) Proc Natl Acad Sci USA 97, 7130-7135
11. Garcia-Cozar, F. J., Okamura, H., Aramburu, J. F., Shaw, K. T., Pelletier, L., Showalter, R., Villafranca, E., and Rao, A. (1998) J Biol Chem 273, 23877-23883
12. Graef, I. A., Chen, F., Chen, L., Kuo, A., and Crabtree, G. R. (2001) Cell 105, 863875
13. Zhang, Z. and Wood, W. I. (2003) Bioinformatics 19, 307-308
14. Iacobuzio-Donahue, C. A., Ryu, B., Hruban, R. H., and Kern, S. E. (2002) Am J Pathol 160, 91-99
15. Corbit, K. C., Trakul, N., Eves, E. M., Diaz, B., Marshall, M., and Rosner, M. R. (2003) J Biol Chem 278, 13061-13068
16. Corbit, K. C., Foster, D. A., and Rosner, M. R. (1999) Mol Cell Biol 19, 4209-4218
17. Bukanov, N. 0., Meek, A. L., Klinger, K. W., Landes, G. M., and Ibraghimov-Beskrovnaya, O. (2000) Funct Integr Genomics 1, 193-199
18. Uehara, Y., Mori, C., Noda, T., Shiota, K., and Kitamura, N. (2000) Genesis 27, 99-103
19. O'Reilly, M. S., Holmgren, L., Shing, Y., Chen, C., Rosenthal, R. A., Moses, M., Lane, W. S., Cao, Y., Sage, E. H., and Folkman, J. (1994) Cell 79, 315-328
20. Chen, F., and Capecchi, M. R (1999) Proc Natl Acad Sci USA 96, 541-546
21. Hennighausen, L., and Robinson, G. W. (1998) Genes Dev 12, 449-455
22. Perou, C. M., Sorlie, T., Eisen, M. B., van de Rijn, M., Jeffrey, S. S., Rees, C. A., Pollack, J. R., Ross, D. T., Johnsen, H., Akslen, L. A., Fluge, 0., Pergamenschikov, A., Williams, C., Zhu, S. X., Lonning, P. E., Borresen-Dale, A. L., Brown, P. 0., and Botstein, D. (2000) Nature 406, 747-752
23. Turner, M. D., Handel, S. E., Wilde, C. J., and Burgoyne, R. D.: (1993) J Cell Sci 106 (Pt 4), 1221-1226
24. Mailleux, A. A., Spencer-Dene, B., Dillon, C., Ndiaye, D., Savona-Baron, C., Itoh, N., Kato, S., Dickson, C., Thiery, J. P., and Bellusci, S. (2002) Development 129, 53-60
25. Grossman, C. E., Qian, Y., Banki, K., and Perl, A. (2003) J Biol Chem
26. Long, W., Wagner, K. U., Lloyd,: K. C., Binart, N., Shillingford, J. M., Hennighausen, L., and Jones, F. E. (2003) Development 130, 5257-5268
27. Srinivasan, K., Strickland, P., Valdes, A., Shin, G. C., and Hinck, L. (2003) Dev Cell 4, 371-382
28. Dontu, G., Al-Hajj, M., Abdallah, W. M., Clarke, M. F., and Wicha, M. S. (2003) Cell Prolif 36 Suppi 1, 59-72
29. Dontu, G., Abdallah, W. M., Foley, J. M., Jackson, K. W., Clarke, M. F., Kawamura, M. J., and Wicha, M. S. (2003) Genes Dev 17, 1253-1270
30. Alberg, A. J. and Helzlsouer, K. J. (1997) Curr Opin Oncol 9, 505-511
31. Hill, A. D., Doyle, J. M., McDermott, E. W., and O'Higgins, N. J. (1997) Br J Surg 84, 1334-1339
32. Nicoletto, M. O., Donach, M., De Nicolo, A., Artioli, G. Banna, G., and Monfardini, S. (2001) Cancer Treat Rev 27, 295-304
33. Shin, B. K., Wang, H., and Hanash, S. (2002) J Mammary Gland Biol Neoplasia 7, 407-413
34. Vu, T. H., and Werb, Z. (2000) Genes Dev 14, 2123-2133
35. Dancey, J. E. (2003) Cancer Biol Ther 2, 601-609
36. Mitchell, K. J., Pinson, K. I., Kelly, O. G., Brennan, J., Zupicich, J., Scherz, P., Leighton, P. A., Goodrich, L. V., Lu, X., Avery, B. J., Tate, P., Dill, K., Pangilinan, E., Wakenight, P., Tessier-Lavigne, M., and Skarnes, W. C. (2001) Nat Genet 28, 241-249
37. Neilson, J. R., Winslow, M. M., Hur, E. M., and Crabtree, G. R. (2004) Immunity, in press
38. Diel I. J. and Cote R. J. (2000) Cancer Treat Rev 26, 53-65.
39. Chang, C. P., Neilson, J. R., Bayle, H., Gestwicki, J. E., Kuo, A. C., Graef, I., and Crabtree, G. R. (2004) Submitted.

### INCORPORATION BY REFERENCE

All of the publications cited herein are hereby incorporated by reference in their entirety to describe more fully the art to which the application pertains.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for augmenting diagnosis of a cell-proliferative disorder comprising detecting the presence of a CRKD marker in a biological sample obtained from a patient, wherein the presence of said marker is indicative of cancer.

2. The method of claim 1, wherein said cell-proliferative disorder is breast cancer.

3. The method of claim 1, wherein said CRKD marker is a CRKD polypeptide.

4. The method of claim 3, wherein said CRKD polypeptide is encoded by a nucleic acid comprising SEQ ID NO:1 or SEQ ID NO: 3 or a fragment thereof, or comprises the amino acid sequence of SEQ ID NO: 2 or SEQ ID NO: 4 or a fragment thereof.

5. The method of claim 3, wherein said fragment comprises the extracellular domain of a CRKD polypeptide.

6. The method of claim 1, wherein said sample is a breast tissue sample.

7. The method of claim 1, wherein said sample is a body fluid sample.

8. The method of claim 7, wherein said body fluid sample is blood or serum.

9. The method of claim 1, wherein said CRKD marker is a nucleic acid.

10. An isolated antibody or fragment thereof which binds specifically to a CRKD polypeptide.

11. The antibody or fragment thereof of claim 10, wherein said CRKD polypeptide is encoded by a nucleic acid comprising SEQ ID NO:1 or SEQ ID NO: 3 or a fragment thereof, or comprises the amino acid sequence of SEQ ID NO: 2 or SEQ ID NO: 4 or a fragment thereof.

12. The antibody or fragment thereof of claim 10, wherein said antibody is a CRKD antagonist.

13. An isolated antibody or fragment thereof which binds specifically to a CRKR polypeptide.

14. The antibody or fragment thereof of claim 13, wherein said CRKR polypeptide is encoded by the nucleic acid sequence of SEQ ID NO:5 or a fragment thereof, or comprises the amino acid sequence of SEQ ID NO:6 or a fragment thereof.

15. The antibody or fragment thereof of claim 13, wherein said antibody is a CRKD antagonist.

16. A method of monitoring the effectiveness of a treatment against a cell-proliferative disorder in which CRKD is upregulated, comprising quantifying the amount of a CRKD marker in a biological sample, wherein a decrease in the CRKD marker is indicative of the effectiveness of the treatment.

17. A method of treating a cell-proliferative disorder in which CRKD is upregulated comprising administering to a mammal an effective amount of pharmaceutical composition comprising a CRKD antagonist.

18. A method for identifying the presence of mammary stem cells in a mixed cell population, comprising detecting the presence of a CRKD marker, wherein the presence of CRKD polypeptide is indicative of the presence of mammary stem cells in a mixed cell population.

19. A method for isolating mammary stem cells comprising:
a) obtained a mixed cell population;
b) exposing said mixed cell population to a binding moiety specific for a CRKD marker; and
c) separating the cells bound to the binding moiety, thereby isolating mammary stem cells.

20. A method to screen for a compound used to treat a cell-proliferate disorder comprising:
a) identifying a CRKD antagonist; and
b) determining whether said CRKD antagonist is effective against a cell-proliferative disorder.


SEQUENCE LISTING (6 sequences)

SEQ ID NO: 1 - DNA, 2539 bp, Mus musculus
tacaatttaaatatattgcttccagacagataattcttagttgtgctgtttatgtggtcttaaattcttcctgcttctgcaatcagcatccgctcttacaactgtgtagtgcttttggggctcctaatccaccccactccacgatgccttctgacacagtgactctgtactcacactggtggaaaaggaagaaaagccctgggcatgcagttgtgcaggtctcacgcggagctgctgctaagcaaaacctctctgccactctcggcttggctgagttcctctcttggctcagggttagtgatctggtaaggtctaggaggcttaggacccagggggcgcctttcagctgaaaaacacctcggctgcagcaagctagctgggaagctcccagttctaaagagaggctgtttaccagaagagcataacaggggccggccagtctgactgcaaggagacaaaactgctgccaaggaggacccccccccccccatttggacactggctgttgagtcacctgcgagccgaagagacgaggatgctgttggcttgggtacacacatttcttctcagcaacatgcttctggcagaagcctatggatctggaggctgcttctgggacaacggccacctgtaccgggaggaccagccctcgcccgcgccgggtctccgctgcctcaactggttggccgcgcaaggcagccgcgagtcgctcaccgagcccagcccgggcaaccacaactactgccggaacccggaccaggacccgcgcgggccctggtgctacatcagcagcgagaccggcgtccctgaaaagcggccctgcgaggacgtgagttgcccagagaccacttcccaagcaccaccgccatcctctgccatggagctggaagagaagtctggtgcaccaggtgacaaagaggcacaggtgttccctcctgctaacgccctaccagccaggagtgaggcagccgaggtgcagccagtgatcgggatcagtcagcttgtgaggatgaactccaaggaaaaaaaagacctaggaactctgggctacgtgctgggcattactatgatggtgatcatcctcgctattggagctggcattatcgtgggctacacttacaagagggggaaggacttgaaagagcaacatgagaagaaagcgtgtgagagggagatgcagcgaatcaccttgcccctgtctgccttcacaaaccccacctgtgagaccgtggatgaaaacaccatcattgtgcacagcaaccagactcctgctgacgtgcaggagggcagcaccctcctcacgggccaggctggcacccctggggcctgagccccttcagtgggcaggtgcccatgtaaacactggtacaggacaggccaccctcctgcagttgggaggagctacattttgtgttgtggttaaaaccttatcccatttttcttttggggggaatgtgactcctcatgacacaagaagatgcaggtagcattatgggtgagggtgaggaggctgggtagggtcctatcaacgctccttttctatcccttggagcagaatttgcctgcggagagagactggcacagtagccacagtggtgctgtggcaaataagggcttccacattgcctgggccaaggaaagactgggagctggctaaagctcttctgtcctgtatactgaagaaggtggcctcagtctctgcttttccattcgtcatccaggaacttgagtggcatatactgttattcctagttaaggtcaagcataaaaaaaactatatatatataaatgaaagaaatatttaggatgaaagtcccttctgacttctaagaacctgaaatgaatctgcttgctggcagcgatgtgccttgacaactgtgggataggcctgggcccgagggtctcttcctatctcgtaaggaaagggaagaattgcataaactttccacttaggtgaaggacccgcttctgccttcagcctcatgcagcagaccagaatatccagactgtcttttgggggctggatagaggaacctctcaattttgcgctgataagaaaatagccaattcatgggaattacacacttgagagtgtggtgacccgcagctgaaaggtgagcttggagaccaagccagaaggttggtaacatgctggggcttgcgttgtagtcctgccgggcttgcaggccagttccttccatagggttaagattaactggtacaaaacaatcactgcaccactgcgaaagacctgaaggagttccagaaagggcaaagcagcttcccagtgtcctcctggccttggcagtgtccccagccaggagcccttccactcccaacactgggagcaggcttcaccaggctgctctcataagccatttgttgagggggcctggatctcagggttcccatggtggtggtgactgtgggaagagagcagtactacttcctactttcctatgaaagtatttctctgtatgttttatatacctcattctgacacctgtgtatgaactatgcaatttgttttgcttttgac

SEQ ID NO: 2 - protein, 264 aa, Mus musculus
MLLAWVHTFLLSNMLLAEAYGSGGCFWDNGHLYREDQPSPAPGLRCLNWLAAQGSRESLTEPSPGNHNYCRNPDQDPRGPWCYISSETGVPEKRPCEDVSCPETTSQAPPPSSAMELEEKSGAPGDKEAQVFPPANALPARSEAAEVQPVIGISQLVRMNSKEKKDLGTLGYVLGITMMVIILAIGAGIIVGYTYKRGKDLKEQHEKKACEREMQRITLPLSAFTNPTCETVDENTIIVHSNQTPADVQEGSTLLTGQAGTPGA

SEQ ID NO: 3 - DNA, 2443 bp, Homo sapiens
agttctaaagagaggctgtttaccagaacagcataacaagggcaggtctgactgcaaggctgggactgggaggcagagccgccgccaagggggcctcggttaaacactggtcgttcaatcacctgcaagacgaaggaggcaaggatgctgttggcctgggtacaagcattcctcgtcagcaacatgctcctagcagaagcctatggatctggaggctgtttctgggacaacggccacctgtaccgggaggaccagacctcccccgcgccgggcctccgctgcctcaactggctggacgcgcagagcgggctggcctcggcccccgtgtcgggggccggcaatcacagttactgccgaaacccggacgaggacccgcgcgggccctggtgctacgtcagtggcgaggccggcgtccctgagaaacggccttgcgaggacctgcgctgtccagagaccacctcccaggccctgccagccttcacgacagaaatccaggaagcgtctgaagggccaggtgcagatgaggtgcaggtgttcgctcctgccaacgccctgcccgctcggagtgaggcggcagctgtgcagccagtgattgggatcagccagcgggtgcggatgaactccaaggagaaaaaggacctgggaactctgggctacgtgctgggcattaccatgatggtgatcatcattgccatcggagctggcatcatcttgggctactcctacaagagggggaaggatttgaaagaacagcatgatcagaaagtatgtgagagggagatgcagcgaatcactctgcccttgtctgccttcaccaaccccacctgtgagattgtggatgagaagactgtcgtggtccacaccagccagactccagttgaccctcaggagggcaccaccccccttatgggccaggccgggactcctggggcctgagcccccccagtgggcaggagcccatgcagacactggtgcaggacagcccaccctcctacagctaggaggaactaccactttgtgttctggttaaaaccctaccactcccccgcttttttggcgaatcctagtaagagtgacagaagcaggtggccctgtgggctgagggtaaggctgggtagggtcctaacagtgctccttgtccatcccttggagcagattttgtctgtggatggagacagtggcagctcccacagtgatgctgctgctaagggcttccaaacattgcctgcacccctggaactgaaccagggatagacggggagctcccccaggctcctctgtgctttactaagatggcctcagtctccactgtgggcttgagtggcatacactgttattcatggttaaggtaaagcaggtcaagggatggcattgaaaaaatatatttagtttttaaaatatttgggatggaactccctactgacctctgagaactggaaacgagtttgtacagaagtcagaactttgggttgggaatgagatctaggttgtggctgctggtatgcttcagcttgctggcaatgatgtgccttgacaaccgtgggccaggcctgggcccagggactcttcctgtttcataaggaaaggaagaattgcactgagcattccacttaggaagaggatagagaaggatctgctccgcctttggccacaggagcagaggcagacctgggatgccccagtttctcttcagggatggatagtgacctgtcttcattttgcacaggtaagagagtagttagctaacctatgggaattatactgtggggccttgtgagctgcttctaagaggctaacctggaaactaagctcagaggcaaggtaataaagcacttcagggcttgctccccaagtgggcctgatttagcaggtggtcctgcgggcgtccaggtcagcaccttcctgtagggcactggggctagggtcacagcccctaactcataaagcaatcaaagaaccattagaaagggctcattaagccttttggacacaggaccccagagaggaaaaagtgacttgcccaaggtcgtaagcaagctactggcatggcaagagcccagcttcctgacggagcgcaacatttctccactgcactgtgctagcagctcagcagggcctctaacctgtgatgtcacactcaagaggccttggcagctcctagccatagagcttcctttccagaacccttccactgcccaatgtggagacgggttagtggggctttctatggagccatctgctttggggacctagacctcaggtggtctcttggtgttagtgatgctggagaagagaatattactggtttctacttttctataaaggcatttctctatatacatgttttatatacctcattctgacacctgcatatagtgtgggaaattgctctgcatttgacttaattaaaaaaaaaaaagactccaaaaaaaaaaaaaaaaaa

SEQ ID NO: 4 - protein, 263 aa, Homo sapiens
MLLAWVQAFLVSNMLLAEAYGSGGCFWDNGHLYREDQTSPAPGLRCLNWLDAQSGLASAPVSGAGNHSYCRNPDEDPRGPWCYVSGEAGVPEKRPCEDLRCPETTSQALPAFTTEIQEASEGPGADEVQVFAPANALPARSEAAAVQPVIGISQRVRMNSKEKKDLGTLGYVLGITMMVIIIAIGAGIILGYSYKRGKDLKEQHDQKVCEREMQRITLPLSAFTNPTCEIVDEKTVVVHTSQTPVDPQEGTTPLMGQAGTPGA

SEQ ID NO: 5 - DNA, 3465 bp, Mus musculus
gagctctcctcaggcctggccttgtgaggcaggagatcagagtctctgagagctttcatctagttcagagcagggctgtagctggaggatccacaggactgcacaggccttgcccagcttctctctcatccacagaggtctctgccttccttagcagctcctgcagaactggctgtggtggctcctgaatcctgcccagggttgctcttctctcttcagagcgcccctcctttcttgctcaacatcacagctcaggaggactcagtgatggcctgggagcccacatacctgctctccccagtgctgctgctgctcctggcctcaggctcctggacacagaaaccagagttacttcgagcacaggagggtgagactgtttctttgacatgctggtatgattcgctctaccactccgatgagaagatctggtgtaagcaaatagacaacttgtgttacctcttcgtcagcaaaagtgccgagaagccaagattcctcatccagcagtcttctcgcttcaacttcttcactgtcaccatgactaagctcaagatgagtgactcgggcatctatcactgtgggatcgctgtaaataccaggataatttatctcagaagtattcacctggtggtgtcaaaagcttcatccacaaccacctggaggacaacaaccctggcctctacccacagccccgtcactaacagaagctttccagatagcccgatgtggaaggccatcgttgctggggtggtcgtggctgttctgctactgctcacattcgtcatccttgtgatcctgtacctgaggaaagctcgaagaaaggccctgaacgttcagaaccaatgtcaccctatctatgaagacttttcagaccagaaggaggagaccactagcttcaatcagcagacccactccagtgaggacactgggaccatctgctatgcctctctcatccacctaaaccgtgtaaaccctcaggactccatctacagcaacacccagccctacccgaagccctctcctgacccacttctgactgtggaatatgccagcatctctagaaacagactcggctcttccaagccagattacccaagaggtgaagaccagcaactgagggctgagctcccagggcagtgagcatgtaggtacaacgctactgtccctgtggaggggcaacttcctacacagagttaagatggcctcagatgacagaggacacactttcttcaaggaaggtgactgaaagtgccatccagaaggactctggagccctgttctcacccttctggaggcctaggaccaatgctgctaacacattctgtgtggaagagagatgggtcggcgcctctgagtgaccttaacctcccttccttgtgtgaccaccacttccttcccccctggttcatgaaatcgaagccggttgcacaagttcctcaggaactttaggcagaactatgacattctgcccaacccccaatccctcccacaccagcctgacccttgtttctaaaaccatcctgctcagggaactctggtgaccttgctaggatgaatactaagatgtaggcggtgatcttctgaggtatttccaaccatgtcaaaagtgggaacttccttaccatagccagccacacatctctgtcagaacacctgggctccccggccttctagaggactagcattcactctgcaaactgtgtctacctgtaccacctaagaccacacgctccttccagggtcctagcactgaagatgctgttcctggacgcggactgctcacccctatgaggcagacccttgggggggatggggtccctatgaggcagacccccggggagcagggagatgcagctacagagaaggaagggatgctgtgagtgtggccctgttcctgagcactctgcctcaccctgcctgcccatgccctcctcctatgatgcagatttgcctgggggagacaggatgcttaccacctccagcctcacacccaactccatctcgctcctgggttattggtctctgctcctgatttcagttgacctgtggtccttcgtgcctcctaactgcagccgtgctcttgggccatgcttcagtcaacaccgtctcctggaagcctgctttctattgtacattccttgaattgctcttcttggctatggctcatcctgaaggttgggcttctcatgagctgtcaaaacccaaaagggtttttcagactggagctcctggtgctgactaagcagtattcgctgggtcagtatcaatagatacttaggactcccaggacctacactcaagcttgaggacaagagagattgattgtaggatgtagttgggaatacttataatgtgtccctttgcctctatatggatgaagagcctaaacgctcctatttacatgcattcctccattttacagatggagatatactgaggcacggggagacttggtctcacaggtaataaacggtaaattgccgatgatgttgatttttgccactgcacttgtggttcctgctccctcacggatctctttgtgtatgtgctgggggagacatctgaagggacagtggaaatcaaggcttagatatagggatacactaggggagtgggacccgagcgacagaggcctcaggatgcaacagaggacaacagtgcagcttacactgctgagacggtggaggcgagactgagccggttctagacatccgctaccactgaaggctccgaggcctctccagagcgtcttgtcacctcttcttagctcaggcctctgcagcaagccagcaccctgtggccaggggtgtctgaggagaaaagaggcgtctcatttcctgttgctgtgataaaataccatgagaaaaaagaacttacatcagcaaagggtgagttgaacttacagtcagagggtacattccacaacagttgggaagtcctggcagcaggagccggaggcagctcgccatgttacatctcagtcagtaagcaggcagtggtgatggctggtgctcaacttcctctctccttgtttgtgtgtgtctgtgtgtgtgtgtggtgtgtgtgtgtgtgtgtgtgtgtgtgtctaggtggcacatgcatacaggttcccccctcttggagtccaggacccaaacctaggttacggtttgcctaccttcagggtgagtcttcccacttccattaacctaagctagattgtcgctcacaggcctggacaggggcttgccaccttggtgattctagaccttgtattggccatcccagaggagagacacttcttaaccttttgagctcccagaggttccaacatgtgtttgtgtgtgagctgttcctgagggtcggtcttcattctccctgcctcttgctcagggaaccccaggtgatagctaacctctcctttatgcctcaggggcatctcaggtcacctcagtgagatattcctttgtgtaattccatggcaataac

SEQ ID NO: 6 - protein, 289 aa, Mus musculus
MAWEPTYLLSPVLLLLLASGSWTQKPELLRAQEGETVSLTCWYDSLYHSDEKIWCKQIDNLCYLFVSKSAEKPRFLIQQSSRFNFFTVTMTKLKMSDSGIYHCGIAVNTRIIYLRSIHLVVSKASSTTTWRTTTLASTHSPVTNRSFPDSPMWKAIVAGVVVAVLLLLTFVILVILYLRKARRKALNVQNQCHPIYEDFSDQKEETTSFNQQTHSSEDTGTICYASLIHLNRVNPQDSIYSNTQPYPKPSPDPLLTVEYASISRNRLGSSKPDYPRGEDQQLRAELPGQ